SEQUENCE LISTING (20 sequences)

SEQ ID NO: 1 - DNA, 2334 bp, Aspergillus nidulans
  CDS (400)..(445)
  CDS (499)..(774)
  CDS (828)..(1882)
ttagccctccctttccgatcctatggttcctccgatcctgaccctggttgaccagtgatccggggccttcgagcctttccaccgtgccagccgactttcccgccagattttagtcgcctcccgtggccgcacccagggatcttctcgtccgactccgggcagtatactcattcgccggcctccgccaggccgccgtccgcctctccccaccacccgtctcctctgtcatacttaaacccggtcgtgtgcctctcttggacggtctctttttttttcttatccatcagagccctttgcttctctgctgcttcaagtgcttcaattgtcttctcgcgggctcagcgcattcttattacctttgccacattaccttatcagcaataagcttctcgcgcaaaaatgtctaaccttcccgttgagcccgagttcgagcaggcctacaagggtacgaccaccgttaatgaatggccaatgatcacagctaatgttggcgcgcagagcttgcgtcgaccctcgagaactccaccctctttgagcagcaccctgaataccgacgggctctccaggtcgtctccgttcccgagcgcgttatccagttccgtgtcgtttgggagaacgacaagggcgaggttcagatcaaccgcggttaccgtgttcagttcaactccgctctcggtccctacaagggtggtctccgtttccacccctccgtcaacctttctatcctgaagttccttggcttcgagcagatcttcaaaaatgctctcacaggacgtgcgtaaccgttacttcattggatgtttgccaagagtactaattggtattagtaaacatgggtggtggcaagggtggttccgacttcgaccccaagggcaagtctgactctgaaattcgtcgcttctgtaccgctttcatgactgagctctgcaagcacatcggcgcggacactgaccttcccgctggtgatatcggtgttactggccgtgaggttggtttccttttcggccagtaccgcaggatccgcaaccagtgggagggtgttctcactggcaagggtggcagctggggtggtagcttgatccgccctgaagccactggatacggtgttgtctactacgttcagcacatgatcaagcacgttaccggtggaaaggagtccttcgcaggcaagcgtgtcgccatctccggctccggtaacgttgcccagtacgccgctctcaaggtcatcgagctcggtggttccgttgtctccctttccgactccaagggctctctcattgtcaaggatgagtccgcttctttcacccctgaagagatcgccctcattgccgacctcaaggttgcccgcaagcaactctccgagctcgccacctcctccgctttcgccggcaagttcacctacatccccgatgctcgcccttggaccaacattcccggcaagttcgaggttgctctcccttctgccactcagaacgaagtctccggcgaggaagccgagcacctcatcaagtccggtgtccgctatattgctgagggttccaacatgggttgcacccaggccgccatcgacatctttgaggctcaccgcaacgccaaccccggcgatgccatctggtacgcccctggtaaagccgccaacgctggtggtgtcgccgtctctggtcttgagatggctcagaactctgctcgtctctcctggacatccgaggaggtcgatgctcgcctcaagggcatcatggaggactgcttcaagaacggtctcgagactgctcagaagttcgctactcctgccaagggcgtcctgccttccctcgtcaccggttccaacattgccggtttcaccaaggtcgccgaggccatgaaggaccagggtgactggtggtgaattagccccgtctcctaattttgatgttgcatgatgatgattcctacgtttgttccagcctttcgggttttggtcttgttatgatcttaccattcttagcgtggatagaggcgctggattcgtttctactatgcttttacctgtagatagaccttttttgcatagttatagtatattataccatagacgactttattcttacccttttccttagtaatgtgcaagcactatccacattctgaatagcccagtatgagatttgtactcaaaaaaacttaatgcccagaaaacactcgcaataagcacggccgtgtcgcgttgtgttccttgagcctcaacagttcgaattaattatcgtggttggctcaggcctttatcatgtgactagactaaggattcacgtgattagctcttgcttccacgcttcggaatctacatgaatgaagctt

SEQ ID NO: 2 - protein, 459 aa, Aspergillus nidulans
MSNLPVEPEFEQAYKELASTLENSTLFEQHPEYRRALQVVSVPERVIQFRVVWENDKGEVQINRGYRVQFNSALGPYKGGLRFHPSVNLSILKFLGFEQIFKNALTGLNMGGGKGGSDFDPKGKSDSEIRRFCTAFMTELCKHIGADTDLPAGDIGVTGREVGFLFGQYRRIRNQWEGVLTGKGGSWGGSLIRPEATGYGVVYYVQHMIKHVTGGKESFAGKRVAISGSGNVAQYAALKVIELGGSVVSLSDSKGSLIVKDESASFTPEEIALIADLKVARKQLSELATSSAFAGKFTYIPDARPWTNIPGKFEVALPSATQNEVSGEEAEHLIKSGVRYIAEGSNMGCTQAAIDIFEAHRNANPGDAIWYAPGKAANAGGVAVSGLEMAQNSARLSWTSEEVDARLKGIMEDCFKNGLETAQKFATPAKGVLPSLVTGSNIAGFTKVAEAMKDQGDWW

SEQ ID NO: 3 - DNA, 2571 bp, Aspergillus awamori
  CDS (741)..(785)
  CDS (850)..(1142)
  CDS (1472)..(2243)
tctagattgcgacggcgtattgcttatccttagtaggactccctaatggattccgagcaagaaaagactgtttggcgtgtaccaatggctcatagtaccagcaagagaagaattttctctctcgcttcgagaaagcaatcaaaaaaaaatcctatcctaccctaccctaccctaatacttccattgccacccgattcctcccgatagtagagcgggcgactgccatttggcgggcgggccagcggattcccgccgatagataacgggcagattctgtgacctcaaactatcgactaacagcccgaacttcggcggccaccgccaaacccgccccggaagccggcctcatttgccgtttgggcgtgccaggaaatgccgcctgcagcggagactccctagtgtggtctgtgttgcctgtgtcgtctgtgtagtatactagttactagtctactactgtacagtggatggcctgagggggggactttatgtccgactccggctgttctcctccctctatccactctaccctcttccctctcttctgtctttctccccgctctcgcccctcccctcctcgaaaacataaatcggcctttccccctcgccatcttcttcttcttctccctctcctttctctttcttcttcagactacttctctttctttcatcttttctctatattcctgttttcctagataccccagttaaaaaagttctctcaatcaatcctccccttcagaatgtctaaccttcctcacgagcccgagttcgagcaggcctacaagggtatgttccattgcccctccgaaattgatgatggaaaaaaaattctaacaacatcctcttacagagcttgcctcgacccttgagaactccaccctcttccagaagaaccccgaataccgcaaggcccttgctgtcgtctccgtccccgagcgtgtcatccagttccgtgtcgtctgggaggatgatgccggcaacgtccaggtcaaccgcggtttccgtgtccagttcaacagcgccctcggtccctacaagggtggtcttcgtttccacccctccgtcaacttgtccatcctccaagttccttggtttcgagcagatcttcaagaatgctctcactggcctgaacatgggtggtggtaagggtggttccgacttcgaccccaagggcaagtccgacaacgagatccgtcgcttctgtgtttccttcatgaccgagctctgcaagcacatcggtgccgacactgatgttcccgctggtgacatcggtgtcaccggtcgtgaggtcggtttcctcttcggccagtaccgcaagatccgcaaccagtgggagggtgttctcaccggtaagggtggcagctggggtggttccctcatccgccctgaggccaccggttacggtgttgtctacgtatgtcaattcctcttcttatgattatctatgtataacagcgactaacgcgtaacagtacgtcgagcacatgattgctcacgccaccaacggccaggagtccttcaagggcaagcgcgttgccatctccggttccggtaacgttgcccagtacgccgccctcaaggtcattgagctcggcggttccgtcgtctccctgagcgacacgcagggctccctcatcatcaacggcgagggtagcttcacccccgaggagatcgagctcatcgctcagaccaaggtcgagcgcaacgagctcgccagcatcgtcggtgctgctcccttcagcgacgccaacaagttcaagtacattgctggtgcccgcccctgggttcacgtcggcaaggtcgacgtcgctctcccctccgctacccagaacgaagtttccggcgaggaggcccaggtcctcatcaacgctggctgcaagttcatcgccgagggttccaacatgggttgcacccaggaggccatcgacaccttcgaggcccaccgtaccgccaacgctggcgcggctgccatctggtacgcccccggtaaggccgccaacgccggtggtgtcgctgtctccggtctggagatggctcagaactctgcccgcctcagctggacttctgaggaggttgatgcccgtcttaaggacatcatgcgcgactgcttcaagaacggtcttgagactgctcaggagtacgccacccccgctgagggtgtcctgccttccctggtgaccggatccaacattgccggtttcaccaaggtggctgccgccatgaaggaccagggtgactggtggtaaatgcggaaagccgcaaacccccgcggcttatgtcatgacgattatgtagtttgatgttccctttcagcgcggatggatagaggcgccggtgttttcttgctagtttagatggatgcataatgatatccttttcttaatcctcaaattcttgtaatttgttgtatcaatagtagataatacaactgtagtcaactacccttgcatcttcactatttgcagatgcattcatctctattccgagcacatgcacaaacccatgggaccgcagttcactagtacttagcctgttatcttccctctatcgcatcttaaacaactatctaga

SEQ ID NO: 4 - protein, 370 aa, Aspergillus awamori
MSNLPHEPEFEQAYKELASTLENSTLFQKNPEYRKALAVVSVPERVIQFRVVWEDDAGNVQVNRGFRVQFNSALGPYKGGLRFHPSVNLSILQVPWFRADLQECSHWPEHGWWYVEHMIAHATNGQESFKGKRVAISGSGNVAQYAALKVIELGGSVVSLSDTQGSLIINGEGSFTPEEIELIAQTKVERNELASIVGAAPFSDANKFKYIAGARPWVHVGKVDVALPSATQNEVSGEEAQVLINAGCKFIAEGSNMGCTQEAIDTFEAHRTANAGAAAIWYAPGKAANAGGVAVSGLEMAQNSARLSWTSEEVDARLKDIMRDCFKNGLETAQEYATPAEGVLPSLVTGSNIAGFTKVAAAMKDQGDWW

SEQ ID NO: 5 - DNA, 27 bp, Artificial
  PCR primer
tctagaatgtctaacccccttgttgag

SEQ ID NO: 6 - DNA, 27 bp, Artificial
  PCR primer
gagctctcaccaccagtcaccctggtc

SEQ ID NO: 7 - DNA, 26 bp, Artificial
  PCR primer
ctgcagatgaatgctttagcagcaac

SEQ ID NO: 8 - DNA, 26 bp, Artificial
  PCR primer
tctagataaaccaagaagcctagctg

SEQ ID NO: 9 - DNA, 26 bp, Artificial
  PCR primer
tctagaatgaatgctttagcagcaac

SEQ ID NO: 10 - DNA, 31 bp, Artificial
  PCR primer
gggaaggtttagacattaaaccaagaagcct

SEQ ID NO: 11 - DNA, 30 bp, Artificial
  PCR primer
aggcttcttggtttaatgtctaaccttccc

SEQ ID NO: 12 - DNA, 26 bp, Artificial
  PCR primer
gagctcttacgcctcccatcctcgaa

SEQ ID NO: 13 - DNA, 27 bp, Artificial
  PCR primer
tctagaatgtctaacccccttgttgag

SEQ ID NO: 14 - DNA, 27 bp, Artificial
  PCR primer
gagctctcaccaccagtcaccctggtc

SEQ ID NO: 15 - DNA, 21 bp, Artificial
  PCR primer
cccctcggtatccaattagag

SEQ ID NO: 16 - DNA, 24 bp, Artificial
  PCR primer
cggggggtgggcgaagaactccag

SEQ ID NO: 17 - DNA, 1380 bp, Aspergillus nidulance
atgtctaaccttcccgttgagcccgagttcgagcaggcctacaaggagcttgcgtcgaccctcgagaactccaccctctttgagcagcaccctgaataccgacgggctctccaggtcgtctccgttcccgagcgcgttatccagttccgtgtcgtttgggagaacgacaagggcgaggttcagatcaaccgcggttaccgtgttcagttcaactccgctctcggtccctacaagggtggtctccgtttccacccctccgtcaacctttctatcctgaagttccttggcttcgagcagatcttcaaaaatgctctcacaggactaaacatgggtggtggcaagggtggttccgacttcgaccccaagggcaagtctgactctgaaattcgtcgcttctgtaccgctttcatgactgagctctgcaagcacatcggcgcggacactgaccttcccgctggtgatatcggtgttactggccgtgaggttggtttccttttcggccagtaccgcaggatccgcaaccagtgggagggtgttctcactggcaagggtggcagctggggtggtagcttgatccgccctgaagccactggatacggtgttgtctactacgttcagcacatgatcaagcacgttaccggtggaaaggagtccttcgcaggcaagcgtgtcgccatctccggctccggtaacgttgcccagtacgccgctctcaaggtcatcgagctcggtggttccgttgtctccctttccgactccaagggctctctcattgtcaaggatgagtccgcttctttcacccctgaagagatcgccctcattgccgacctcaaggttgcccgcaagcaactctccgagctcgccacctcctccgctttcgccggcaagttcacctacatccccgatgctcgcccttggaccaacattcccggcaagttcgaggttgctctcccttctgccactcagaacgaagtctccggcgaggaagccgagcacctcatcaagtccggtgtccgctatattgctgagggttccaacatgggttgcacccaggccgccatcgacatctttgaggctcaccgcaacgccaaccccggcgatgccatctggtacgcccctggtaaagccgccaacgctggtggtgtcgccgtctctggtcttgagatggctcagaactctgctcgtctctcctggacatccgaggaggtcgatgctcgcctcaagggcatcatggaggactgcttcaagaacggtctcgagactgctcagaagttcgctactcctgccaagggcgtcctgccttccctcgtcaccggttccaacattgccggtttcaccaaggtcgccgaggccatgaaggaccagggtgactggtggtga

SEQ ID NO: 18 - DNA, 1383 bp, Aspergillus awamori
atgtctaaccttcctcacgagcccgagttcgagcaggcctacaaggagcttgcctcgacccttgagaactccaccctcttccagaagaaccccgaataccgcaaggcccttgctgtcgtctccgtccccgagcgtgtcatccagttccgtgtcgtctgggaggatgatgccggcaacgtccaggtcaaccgcggtttccgtgtccagttcaacagcgccctcggtccctacaagggtggtcttcgtttccacccctccgtcaacttgtccatcctcaagttccttggtttcgagcagatcttcaagaatgctctcactggcctgaacatgggtggtggtaagggtggttccgacttcgaccccaagggcaagtccgacaacgagatccgtcgcttctgtgtttccttcatgaccgagctctgcaagcacatcggtgccgacactgatgttcccgctggtgacatcggtgtcaccggtcgtgaggtcggtttcctcttcggccagtaccgcaagatccgcaaccagtgggagggtgttctcaccggtaagggtggcagctggggtggttccctcatccgccctgaggccaccggttacggtgttgtctactacgtcgagcacatgattgctcacgccaccaacggccaggagtccttcaagggcaagcgcgttgccatctccggttccggtaacgttgcccagtacgccgccctcaaggtcattgagctcggcggttccgtcgtctccctgagcgacacgcagggctccctcatcatcaacggcgagggtagcttcacccccgaggagatcgagctcatcgctcagaccaaggtcgagcgcaacgagctcgccagcatcgtcggtgctgctcccttcagcgacgccaacaagttcaagtacattgctggtgcccgcccctgggttcacgtcggcaaggtcgacgtcgctctcccctccgctacccagaacgaagtttccggcgaggaggcccaggtcctcatcaacgctggctgcaagttcatcgccgagggttccaacatgggttgcacccaggaggccatcgacaccttcgaggcccaccgtaccgccaacgctggcgcggctgccatctggtacgcccccggtaaggccgccaacgccggtggtgtcgctgtctccggtctggagatggctcagaactctgcccgcctcagctggacttctgaggaggttgatgcccgtcttaaggacatcatgcgcgactgcttcaagaacggtcttgagactgctcaggagtacgccacccccgctgagggtgtcctgccttccctggtgaccggatccaacattgccggtttcaccaaggtggctgccgccatgaaggaccagggtgactggtggtaa

SEQ ID NO: 19 - DNA, 1191 bp, Escherichia coli
  CDS (1)..(1188)
atgtttgagaacattaccgccgctcctgccgacccgattctgggcctggccgatctgtttcgtgccgatgaacgtcccggcaaaattaacctcgggattggtgtctataaagatgagacgggcaaaaccccggtactgaccagcgtgaaaaaggctgaacagtatctgctcgaaaatgaaaccaccaaaaattacctcggcattgacggcatccctgaatttggtcgctgcactcaggaactgctgtttggtaaaggtagcgccctgatcaatgacaaacgtgctcgcacggcacagactccggggggcactggcgcactacgcgtggctgccgatttcctggcaaaaaataccagcgttaagcgtgtgtgggtgagcaacccaagctggccgaaccataagagcgtctttaactctgcaggtctggaagttcgtgaatacgcttattatgatgcggaaaatcacactcttgacttcgatgcactgattaacagcctgaatgaagctcaggctggcgacgtagtgctgttccatggctgctgccataacccaaccggtatcgaccctacgctggaacaatggcaaacactggcacaactctccgttgagaaaggctggttaccgctgtttgacttcgcttaccagggttttgcccgtggtctggaagaagatgctgaaggactgcgcgctttcgcggctatgcataaagagctgattgttgccagttcctactctaaaaactttggcctgtacaacgagcgtgttggcgcttgtactctggttgctgccgacagtgaaaccgttgatcgcgcattcagccaaatgaaagcggcgattcgcgctaactactctaacccaccagcacacggcgcttctgttgttgccaccatcctgagcaacgatgcgttacgtgcgatttgggaacaagagctgactgatatgcgccagcgtattcagcgtatgcgtcagttgttcgtcaatacgctgcaggaaaaaggcgcaaaccgcgacttcagctttatcatcaaacagaacggcatgttctccttcagtggcctgacaaaagaacaagtgctgcgtctgcgcgaagagtttggcgtatatgcggttgcttctggtcgcgtaaatgtggccgggatgacaccagataacatggctccgctgtgcgaagcgattgtggcagtgctgtaa

SEQ ID NO: 20 - protein, 396 aa, Escherichia coli
MFENITAAPADPILGLADLFRADERPGKINLGIGVYKDETGKTPVLTSVKKAEQYLLENETTKNYLGIDGIPEFGRCTQELLFGKGSALINDKRARTAQTPGGTGALRVAADFLAKNTSVKRVWVSNPSWPNHKSVFNSAGLEVREYAYYDAENHTLDFDALINSLNEAQAGDVVLFHGCCHNPTGIDPTLEQWQTLAQLSVEKGWLPLFDFAYQGFARGLEEDAEGLRAFAAMHKELIVASSYSKNFGLYNERVGACTLVAADSETVDRAFSQMKAAIRANYSNPPAHGASVVATILSNDALRAIWEQELTDMRQRIQRMRQLFVNTLQEKGANRDFSFIIKQNGMFSFSGLTKEQVLRLREEFGVYAVASGRVNVAGMTPDNMAPLCEAIVAVL

(19) United States
(12) Patent Application Publication
(10) Pub. No.: US 2006/0090219 A1
(43) Pub. Date: Apr. 27, 2006

(54) Methods for producing plants with improved growth under nitrogen-limited conditions

(76) Inventors: Hiroaki Kisaka, Kawasaki-shi (JP); Tetsuya Miwa, Kawasaki-shi (JP); Ai Akiyama, Kawasaki-shi (JP)

Correspondence Address: CERMAK & KENEALY LLP;ACS LLC, 515 EAST BRADDOCK ROAD, SUITE B, ALEXANDRIA, VA, 22314, US

(21) Appl. No.: 11/302,262

(22) Filed: Dec. 14, 2005

Related U.S. Application Data
(63) Continuation of application No. PCT/JP04/10451, filed on Jul. 15, 2004.

(30) Foreign Application Priority Data
Jul. 17, 2003 (JP) 2003-198559

(51) Int. Cl. A01H 1/00; C12N 15/82

(52) U.S. Cl. 800/278; 435/468

(57) ABSTRACT

The present invention provides a method of producing a plant which exhibits improved growth and/or yield under reduced nitrogen conditions, that is, under cultivation conditions where nitrogen is limited as compared to ordinary cultivation conditions, by increasing 2-OG content in plants. Particularly, the present invention provides a method of producing a plant which exhibits improved growth and/or yield under conditions where nitrogen is reduced, that is, under cultivation conditions where nitrogen is limited as compared to ordinary cultivation conditions, by introducing a GDH gene or ECASPC gene into plants and expressing the transgene GDH or ECAPS in the plants, which results in increased 2-OG, or by spraying proline on the leaves of plants to increase the 2-OG content, thereby enhancing the incorporation of nitrogen or metabolic activity of plants. Also provided is a method of cultivating such plants under nitrogen-limited conditions.

The present application claims priority under 35 U.S.C. §119 to Japanese Patent Application No. 2003-198559, filed Jul. 17, 2003, and is a continuation under 35 U.S.C. § 120 of PCT/JP2004/010451, filed Jul. 15, 2004, the entirety of both of which is incorporated by reference. Also, the Sequence Listing on compact disk filed herewith is hereby incorporated by reference (File name: US-257 Seq List; File size: 38 KB; Date recorded: Dec. 13, 2005).

### FIELD OF THE INVENTION

The present invention relates to a method for improving the growth and yields of plants under nitrogen-limited conditions.

### BACKGROUND OF THE INVENTION

Application of chemical fertilizers to supply nitrogen as observed in modern intensive agriculture results in dramatic improvements in yield, but the release of nitrogen into the surrounding environment in the form of excess nitrates raised environmental problems such as contamination of rivers and lakes by eutrophication, health hazards from accumulation of nitrates in crops, and generation of greenhouse gases from residual nitrogen in the soil. The large amount of electricity required for manufacturing nitrogen fertilizers also contributes to the environmental burden of agriculture. In other words, agricultural methods that use less chemical fertilizer, particularly nitrogen fertilizer, are necessary in order to achieve sustainability.

However, with a few exceptions such as the Leguminosae, plants cannot fix nitrogen from the air, and are entirely dependent for nitrogen on nitrate or ammonia supplied from outside. Consequently, nitrogen is the greatest limiting factor on the growth and development of plants, and modern agriculture is faced with the apparently conflicting tasks of maintaining and increasing current yields while reducing the aforementioned environmental burden in an effort towards sustainability. Efforts in this direction, and in particular efforts to develop and cultivate plants with satisfactory growth and yields under conditions of reduced nitrogen supply have so far been inadequate. For example, environment-friendly and sustainable organic agriculture has been proposed as an alternative to modern intensive agriculture, but while application of nitrogen fertilizer is controlled in such environment-friendly organic agriculture, there have been many problems in terms of yield.

In terms of improving plant cultivars, there have been no successful examples of plant breeding to solve these issues by providing adequate growth and/or yields under nitrogen-limited conditions such as the conditions where the application of nitrogen fertilizer is reduced. Consequently, there is a demand for the breeding of plant cultivars having maintained or improved yields by modifying or improving the utilization efficiency of nitrogen by plants under less application of nitrogen fertilizer, and for the development of technologies for breeding such plant cultivars.

The first stage of assimilation of inorganic nitrogen into the organic form in plants is known to be mainly incorporation of ammonia into glutamate to produce glutamine. This is a reaction catalyzed by glutamine synthetase (GS) and glutamate synthase (GOGAT), in which the two reactions combine to produce one molecule of glutamate by assimilation of one molecule of ammonia into one molecule of 2-OG. This GS/GOGAT cycle is thought to be the primary pathway of nitrogen assimilation in plants (Miflin and Lea: 1976, Phytochemistry 15: 873-885).

In microorganisms, on the other hand, it has been reported from studies using Cyanobacteria and E. coli that the quantitative balance of 2-OG and glutamine regulates the expression of enzyme genes associated with incorporation and assimilation of nitrate (Forchammer and Tandeau-Marsac, 1995, J. Bacteriol 177, 2033-2040; Jiang et al, 1998, Biochemistry 37, 12795-12801). However a gene has been isolated from Arabidopsis thaliana (Arabidopsis) having strong homology with the PII protein, which is thought to be associated with such regulation, and when this gene was introduced and over-expressed in tobacco, no increase in nitrate content was seen, suggesting that accumulation of nitrogen in higher plants occurs differently than in microorganisms (Hsieh et al, 1998, Proc. Natl. Acad. Sci. USA 95: 13965-13970). On the other hand, it has been reported that when expression of nitrate reductase (hereinafter abbreviated as NR) was investigated using tobacco plants having the ferredoxin-dependent glutamate synthase gene introduced in the antisense direction so as to reduce expression of glutamate synthase, the transcription level of NR increased when nitrate, sucrose and 2-OG were supplied, while when glutamine was supplied, the NR transcription level decreased (Ferrario-Mery et al, 2001 Planta 213: 265-271). According to this reference, the amounts of 2-OG and glutamine are also associated with regulation of NR in higher plants. However, these findings only relate to NR, and nothing has been said about the effects of 2-OG on the growth and development of plants under nitrogen-limited conditions. Moreover, 2-OG is a relatively unstable compound and has been considered to be impractical for, for example, spraying purposes.

Transgenic plants into which a GDH gene has been introduced have also been developed, and it has been reported that when an E. coli-derived NADP-dependent GDH gene (gdhA) was introduced into tobacco and corn with the aim of conferring resistance to the herbicide phosphinothricin, dried weight, total amino acid content and water-soluble carbohydrate content all increased significantly (Lightfoot et al, CA2180786, 1996). Similarly, Tian et al (CN00109779.2) introduced a Neurospora-derived NADP-dependent GDH gene into tobacco and reported that growth was better than that of tobacco not having the gene introduced. It has also been reported that free amino acid content in the fruit increased when an Aspergillus nidulans-derived NADP-dependent GDH gene was introduced into tomato plants (Kisaka et al, JP11-376710) and that tuber weight and number of tubers increased when the same gene was introduced into potato plants (Kisaka et al, JP2000-404322). However, the function of the introduced gene is not elucidated in any of these reports, and it is unknown how the introduced gene functions or what effects produce these characteristics. Moreover, nothing is mentioned about improving the growth and/or yields of plants under nitrogen-limited conditions.

There have also been no reports regarding introduction of the aspartate aminotransferase gene into plants.

### SUMMARY OF THE INVENTION

It is an object of the present invention to provide a method for developing plants having enhanced nitrogen incorporation and enhanced metabolic activity of plants and having improved growth and/or yields under cultivation conditions of limited nitrogen or in other words with nitrogen limited to the level below the normal cultivation conditions, as well as a method of cultivating such plants under nitrogen-limited conditions.

The inventors assumed the existence of a signal molecule that allows plant cells to sense nitrogen nutrient conditions, and considered 2-oxoglutarate (2-OG) as such a signal molecule. That is, 2-OG is a signal molecule which comprehensively regulates nitrogen incorporation and metabolism in plant cells, and it was thought that by artificially increasing and decreasing the content of this substance it would be possible to maintain nitrogen incorporation and metabolism in an elevated state in plant cells. Moreover, because a plant in such an elevated state is believed to be capable of actively incorporating nitrogen from exterior, it was thought that nitrogen would be replenished under nitrogen-limited conditions, thereby improving growth and yield. Moreover, the inventors discovered that the 2-OG content of plant cells could be increased by causing over-expression of glutamate dehydrogenase (hereunder abbreviated as GDH) activity or aspartate aminotransferase (hereunder abbreviated as ASPC) activity, and that plants with increased 2-OG content have improved growth and/or yields under nitrogen-limited conditions, which led to the establishment of the present inventions.

Consequently, the present invention is a method of producing a plant exhibiting improved growth and/or yield under cultivation conditions where nitrogen is limited to the level below normal cultivation conditions, by increasing the 2-OG content in plants.

Moreover, the present invention is a plant or seed thereof exhibiting improved growth and/or yield under cultivation conditions where nitrogen is limited to the level below normal cultivation conditions, by introducing a GDH gene or ASPC gene and expressing the gene in a plant body, thereby increasing the 2-OG content. Moreover, the present invention is a method for producing a plant exhibiting improved growth and/or yield under cultivation conditions where nitrogen is limited to the level below normal cultivation conditions, by introducing a GDH gene or ASPC gene and expressing the gene in the plant body, thereby increasing the 2-OG content.

In particular, the present invention is a method for producing a plant exhibiting improved growth and/or yield under cultivation conditions where nitrogen is limited to the level below normal cultivation conditions, in which growth and/or yield equivalent to or greater than growth and/or yield obtained under cultivation conditions with standard application amount (defined for each plant) of nitrogen fertilizer is obtained under cultivation conditions with an amount of nitrogen ranging from between below the lower limit and 1/10-fold the lower limit of the standard application amount of nitrogen fertilizer.

The present invention is a method of cultivating a plant produced by the aforementioned method of the present invention, wherein cultivation is under nitrogen-limited conditions. In particular, the present invention is a method of cultivating a plant developed by the aforementioned method of the present invention under conditions of nitrogen fertilizer application in the range between below the lower limit and 1/10-fold the lower limit of the standard application amount (defined for each plant).

Moreover, the present invention is a method of improving growth and/or yields of plants under cultivation conditions with nitrogen limited to the level below normal cultivation conditions, comprising conducting the foliar application of proline.

### BRIEF DESCRIPTION OF THE DRAWINGS

FIG. 1 is a schematic diagram of the constructed plasmid vector, in which Nos-Pro is nopaline synthase promoter; NPTII is neomycin phosphotransferase; Nos-Ter is nopaline synthase terminator; 35S-Pro is cauliflower mosaic virus 35S promoter; Mtd-AN-GDH is an Aspergillus nidulans-derived glutamate dehydrogenase gene (GDH) with an added mitochondrial transit peptide; HPT is hygromycin phosphotransferase; H is HindIII, B is BamHI, X is XbaI, Sp is SpeI, E is EcoRI, LB is left border and RB is right border.

FIG. 2 shows the results of PCR analysis of transformed potato, with A indicating the use of An-GDH gene-specific primers and B indicating the use of NPTII gene-specific primers. Lane 1 is a 100 bp marker, lane 2 is untransformed potato, lane 3 is transformed potato Mtd1, lane 4 is transformed potato Mtd 2, lane 5 is transformed potato Mtd3, lane 6 is transformed potato Mtd 5, and lane 7 is transformed potato Mtd 8.

FIG. 3 shows the results of PCR analysis of a transformed Arabidopsis thaliana, with A indicating the use of An-GDH gene-specific primers and B indicating the use of NPTII gene-specific primers. Lane 1 is an 100 bp marker, lane 2 is an untransformed Arabidopsis thaliana, lane 3 is a transformed Arabidopsis thaliana Mtd 2, lane 4 is a transformed Arabidopsis thaliana Mtd 3, and lane 5 is a transformed Arabidopsis thaliana Mtd 4.

FIG. 4 shows the results of Northern analysis of transformed potatoes, with A indicating RNA extracted from leaf tissue and B indicating RNA extracted from tubers. The patterns were obtained by ethidium bromide staining of 10 μg of total RNA that had been electrophoresed (lower patterns in both A and B). The full-length An-GDH gene was used as the probe. Lane 1 is an untransformed potato, lane 2 is a transformed potato Mtd1, lane 3 is a transformed potato Mtd 2, lane 4 is a transformed potato Mtd 3, lane 5 is a transformed potato Mtd 5 and lane 6 is a transformed potato Mtd 8.

FIG. 5 shows the results of Northern analysis of a transformed Arabidopsis thaliana. RNA extracted from leaf tissue was used. The patterns (lower patterns) were obtained by ethidium bromide staining of 10 μg of total RNA that had been electrophoresed. The full-length An-GDH gene was used as the probe. Lane 1 is an untransformed Arabidopsis thaliana, lane 2 is a transformed Arabidopsis thaliana Mtd2, lane 3 is a transformed Arabidopsis thaliana Mtd 3 and lane 4 is a transformed Arabidopsis thaliana Mtd 4.

FIGS. 6A through 6C are graphs showing the 2-oxoglutarate (2-OG), urea and nitrate contents of GDH-transformed plants (Arabidopsis thaliana). The vertical axis indicates the moles (nmol) per fresh masses, respectively, with Mtd3-10 representing a transformed Arabidopsis thaliana (strain no. 3) and Columbia representing an untransformed Arabidopsis thaliana (n=3).

FIGS. 6D through 6F are graphs showing 2-oxoglutarate (2-OG), urea and nitrate contents of GDH-transformed plants (potato). The vertical axis indicates the moles (nmol) per fresh masses, respectively; with Mtd-8 representing a transformed potato and the Control representing an untransformed potato (n=3).

FIG. 7 is a graph showing the 2-OG content of Arabidopsis thaliana leaf tissue after 3 weeks of cultivation in PNS medium containing different amounts of KNO₃. The mole numbers (nmol) for each live mass are shown on the vertical axis, with C representing untransformed Arabidopsis thaliana and M representing transformed Arabidopsis thaliana (Mtd 3 strain) (n=3).

FIG. 8 is a graph showing the nitrate content of Arabidopsis thaliana leaf tissue after 3 weeks of cultivation in PNS medium containing different amounts of KNO₃. The moles (nmol) for each live mass are shown on the vertical axis, with C representing an untransformed Arabidopsis thaliana and M representing a transformed Arabidopsis thaliana (Mtd 3 strain) (n=3).

FIG. 9 shows an alignment of higher plant glutamate dehydrogenase amino acid sequences, in which AtGDH1 is Arabidopsis thaliana GDH1, AtGDH2 is Arabidopsis thaliana GDH2, LeGDH is tomato (Lycopersicon esculentum) GDH, NtGDH is tobacco (Nicotiana tabacum) GDH, and ZmGDH is corn (Zea mays) GDH.

FIG. 10 shows an alignment of mold GDH and plant (tomato) GDH amino acid sequences, with AaGDH representing Aspergillus awamori GDH, ANGDH representing Aspergillus nidulans GDH and LeGDH representing tomato GDH.

FIG. 11 is a graph showing the free proline content in leaf tissue 1 and 5 hours after foliar application of an aqueous solution of a spreading agent, urea, and proline (n=6) to potato leaf tissue, respectively, with A indicating 1 hour after application and B indicating 5 hours after application.

FIG. 12 is a graph showing the 2-OG content in leaf tissue 1 hour and 24 hours after foliar application of an aqueous solution of a spreader, urea, and proline (n=6) to potato leaf tissue, respectively, with A indicating 1 hour after application and B indicating 24 hours after application.

FIG. 13 shows the genome PCR analysis results for MtdECASPC transformed Arabidopsis thaliana. PCR analysis was performed using ECASPC-specific primers, in a reaction of 30 cycles consisting of 1 minute at 94° C., 1 minute at 55° C., and 2 minutes at 72° C. Electrophoresis on 1% agarose gel was followed by ethidium bromide staining. Lane 1 is a 1 kbp marker, lane 2 is mtdECASPC plasmid DNA, lane 3 is mtdECASPC transformed Arabidopsis thaliana mtdECASPC2-2, lane 4 is mtdECASPC transformed Arabidopsis thaliana mtdECASPC6-2, lane 5 is mtdECASPC transformed Arabidopsis thaliana mtdECASPC8-1, and lane 6 is mtdECASPC transformed Arabidopsis thaliana mtdECASPC9-1.

FIG. 14 shows RT-PCR analysis of mtdECASPC transformed Arabidopsis thaliana. RNA was extracted from Arabidopsis thaliana leaf tissue using a Qiagen Plant RNeasy Mini-Kit. RT-PCR was performed using a TaKaRa RNA-PCR Kit with ECASPC-specific primers, in a reaction of 30 cycles consisting of 1 minute at 94° C., 1 minute at 55° C., and 2 minutes at 72° C. Electrophoresis on 1% agarose gel was followed by ethidium bromide staining. Lane 1 is a 1 kbp marker, lane 2 is mtdECASPC plasmid DNA, lane 3 is RNA without a reverse transcriptase reaction, lanes 4-5 are untransformed Arabidopsis thaliana, lane 6 is mtdECASPC transformed Arabidopsis thaliana mtdECASPC2-2, lane 7 is mtdECASPC transformed Arabidopsis thaliana mtdECASPC6-2, lane 8 is mtdECASPC transformed Arabidopsis thaliana mtdECASPC8-1, and lane 9 is mtdECASPC transformed Arabidopsis thaliana mtdECASPC9-1.

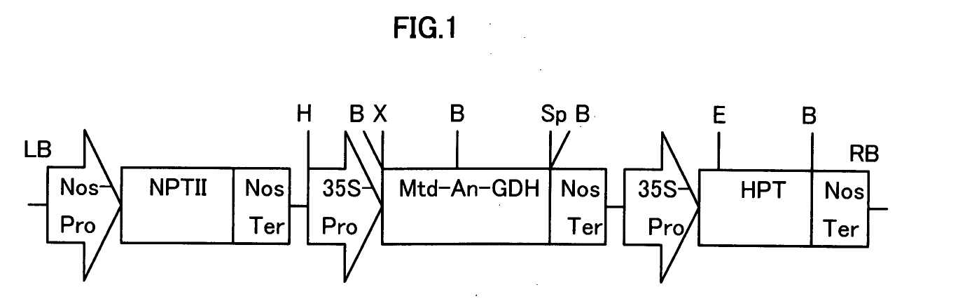

### DETAILED DESCRIPTION OF THE PREFERRED EMBODIMENTS

As described above, the inventors assumed the existence of a signal molecule that allows plant cells to sense nitrogen nutrient conditions. This signal molecule comprehensively regulates nitrogen absorption and metabolism in plant cells, and it was thought that by artificially increasing and decreasing the amount of this substance it would be possible to maintain nitrogen absorption and metabolism in an enhanced state in plant cells. Moreover, because a plant in such an enhanced state is believed to be capable of actively incorporating nitrogen from its exterior, it was thought that nitrogen would be replenished under nitrogen-limited conditions, thus improving growth and yields. Moreover, the inventors supposed 2-oxoglutarate (2-OG) to be this signal molecule.

The first stage of assimilation of inorganic nitrogen into the organic form in plants is mainly the incorporation of ammonia into glutamate to produce glutamine, a reaction which is catalyzed by glutamine synthetase (GS) and glutamate synthetase (GOGAT), and as already mentioned above, this GS/GOGAT cycle is thought to be the primary pathway of nitrogen assimilation in plants. Consequently, the degree of nitrogen assimilation or the degree of nitrogen deficiency in plant cells is thought to reflect the amount of 2-OG.

The inventors propose causing over-expression of glutamate dehydrogenase (GDH) activity or aspartate aminotransferase (ASPC) activity is one possible method of increasing the 2-OG content of plant cells. GDH catalyzes the reversible reactions of incorporating ammonia into 2-OG to produce glutamate and releasing ammonia from glutamate to produce 2-OG. However, it is thought that because, in general, GDH has a high Km value with respect to ammonia, it is more likely to break down glutamate into 2-OG and ammonia than to incorporate ammonia into 2-OG to produce glutamate, so an increase in 2-OG was expected as a result of the reaction. On the other hand, aspartate aminotransferase catalyzes the reaction of transposing the amino groups of oxaloacetate, resulting in the production of aspartic acid and 2-OG, so an increase in 2-OG content was expected.

The inventors transformed plants with the GDH gene or ASPC gene and confirmed an actual increase in the 2-OG content, and also confirmed that the growth and yields of these plants were improved under cultivation conditions where nitrogen is limited to below normal cultivation conditions.

In one embodiment of the present invention, a nucleic acid construct comprising the glutamate dehydrogenase (GDH) gene or aspartate aminotransferase (ASPC) gene is introduced into a plant to obtain a plant which has increased 2-OG content due to the expression of the glutamate dehydrogenase (GDH) gene or aspartate aminotransferase (ASPC) gene in the plant, and which has improved growth and/or yield under cultivation conditions where nitrogen is limited to below normal cultivation conditions.

In another embodiment of the present invention, the copy numbers and/or transcribed amount of an endogenous GDH gene or endogenous ASPC gene is increased, resulting inenhanced GDH or ASPC activity. The 2-OG content of the plant body is also increased, resulting in a plant with improved growth and/or yield under cultivation conditions where nitrogen is limited to below normal cultivation conditions. To increase the transcribed amount, a gene construct capable of expressing a transcription activation factor can be introduced, and/or an enhancer or other cis-functional element can be introduced. Such transcription activation factors and cis-functional elements are well known to those in the field.

There are no particular limits on the origin of the glutamate dehydrogenase used in the present invention, but glutamate dehydrogenase from a microorganism belonging to the genus Aspergillus is preferable. Glutamate dehydrogenase derived from Aspergillus nidulans and Aspergillus awamori is more preferable. Moreover, glutamate dehydrogenase having one or more of its amino acids deleted, substituted, or added can be used in the present invention as long as it has the aforementioned glutamate dehydrogenase activity and selectively catalyzes the reaction of producing 2-OG and ammonia from glutamate under physiological conditions within plant bodies.

There are also no particular limits on the origin of the aspartate aminotransferase used in the present invention, but aspartate aminotransferase derived from Escherichia coli is preferable. Moreover, aspartate aminotransferase having one or more of its amino acids deleted, substituted, or added can be used as long as it has the aforementioned aspartate aminotransferase activity.

Regarding the localization of the glutamate dehydrogenase or aspartate aminotransferase in the cellular organelles, those localized in the mitochondria, chloroplast, peroxisome, or the like, as well as those localized in the cytoplasm can be used. Consequently, a glutamate dehydrogenase or aspartate aminotransferase gene having added to the N- or C-end thereof either a signal or a transit peptide for transferring the enzyme protein to these cellular organelles can also be used in the present invention.

Such a glutamate dehydrogenase gene or its cDNA can be prepared relatively easily by someone skilled in the art based on published sequence data. In the case of the Aspergillus nidulans glutamate dehydrogenase gene, for example, the nucleotide sequence of the genome gene is published under GenBank accession No. X16121. This nucleotide sequence is shown in SEQ ID NO: 1, and the amino acid sequence in SEQ ID NO: 2. Glutamate dehydrogenase cDNA can be obtained easily by synthesizing PCR primers for amplifying DNA fragments so as to include the part encoding the protein based on its sequence, and performing RT-PCR using RNA extracted from cultured cells of Aspergillus nidulans as the template. The cDNA sequence of the Aspergillus nidulans glutamate dehydrogenase gene is shown in SEQ ID NO: 17. Aspergillus nidulans can be obtained, for example, as ATCC10074 or ATCC11267 from the American Type Culture Collection. The genome sequence for the glutamate dehydrogenase gene of Aspergillus awamori is also published under GenBank accession NO: Y15784. This nucleotide sequence is shown in SEQ ID NO: 3, and the amino acid sequence as SEQ ID NO: 4. The cDNA sequence of the Aspergillus awamori glutamate dehydrogenase gene is shown in SEQ ID NO: 18. Aspergillus awamori itself can be obtained from the American Type Culture Collection as ATCC10548 and ATCC11358. Glutamate dehydrogenase cDNA can be obtained by methods similar to those described above based on these strains and sequence information.

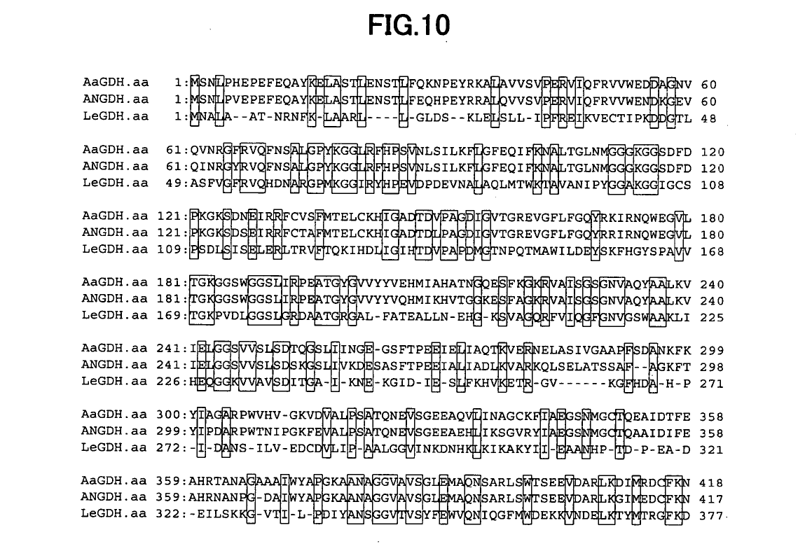

The homology between Aspergillus nidulans GDH and Aspergillus awamori GDH is about 84% in the amino acid sequence (FIG. 10).

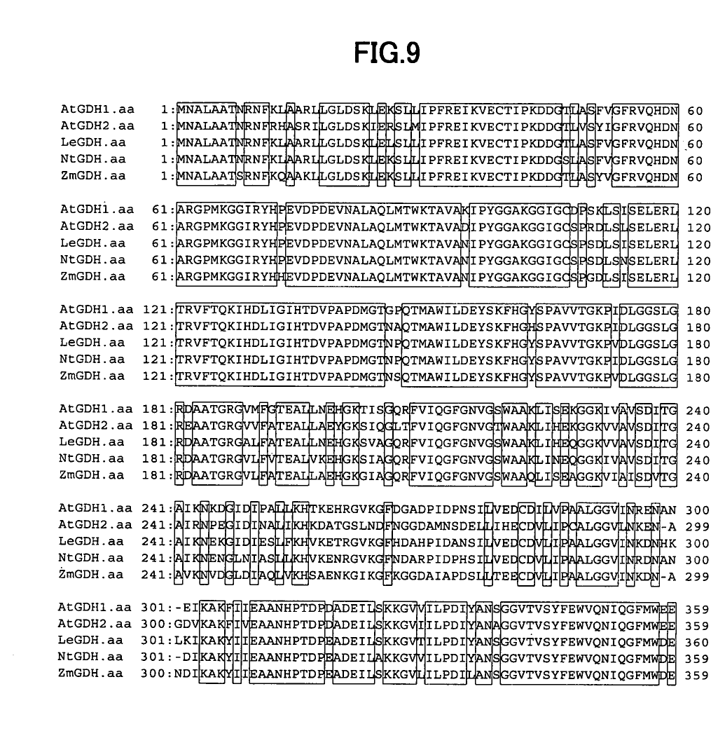

In addition, alignment results for the GDH amino acid sequences of Aspergillus nidulans, Aspergillus awamori, and tomato (Lycopersicon esculentum) are shown in FIG. 10, while alignment results for the amino acid sequences of Arabidopsis thaliana GDH1, Arabidopsis thaliana GDH2, tomato GDH, tobacco (Nicotiana tabacum) GDH, and corn (Zea mays) GDH are shown in FIG. 9.

In the present invention, plants which have improved growth and/or yield under cultivation conditions where nitrogen is limited below ordinary cultivation conditions and which have increased 2-OG content of the plant can be obtained using genes which encode a variety of enzymes which produce 2-OG from glutamate and which are different from the glutamate dehydrogenase gene. Examples of such enzymes include aspartate aminotransferase and alanine aminotransferase.

For example, the nucleotide sequence of genome DNA for the aspartate aminotransferase gene of E. coli strain K-12 is recorded under GenBank accession No. X03629. The aspartate aminotransferase gene can be easily obtained by synthesizing PCR primers based on that sequence so as to amplify a DNA fragment comprising the region encoding the protein, and performing PCR using these primers with chromosome DNA extracted from E. coli strain K-12 as the template.

In the present invention, the growth and/or yields of plants under nitrogen-limited cultivation conditions where nitrogen is limited to below normal cultivation conditions can be improved by applying proline to leaf surfaces, which results in increasing 2-OG content. The proline applied to the leaves is incorporated into the leaf tissue and metabolized into glutamate. Moreover, because this glutamate is metabolized into 2-OG by endogenous glutamate dehydrogenase, it can increase the 2-OG content in the tissue. When the 2-OG content has been increased by application of proline to the leaf surfaces of a plant, the plant will have improved growth and/or yield under conditions in which the amount of available nitrogen during the cultivation period is limited to below ordinary cultivation conditions.

A nucleic acid construct that can be used in the present invention can be prepared using ordinary methods known to those skilled in the art. For example, Sambrook et al, Molecular Cloning—Laboratory Manual, 2^{nd} Ed., Cold Spring Harbor Laboratory Press, or the like, can be consulted with regards to molecular biology techniques, including methods for isolating the nucleic acid construct and determining its sequence. Alternatively, in some cases genetic amplification such as PCR is necessary for preparing a nucleic acid construct which can be used in the present invention, and F. M. Ausubel et al (eds.), Current Protocols in Molecular Biology, John Wiley & Sons, Inc. (1994) can be consulted for such methods.

Nucleic acid constructs which can be used in the present invention may normally include suitable promoters which are able to function in plant cells, such as the nopaline synthase gene or cauliflower mosaic virus 35S promoter (CaMV35S), suitable terminators such as the nopaline synthase gene terminator, other sequences which are necessary or useful for expression, and marker genes for selecting transformants, such as drug resistance genes for kanamycin resistance, G418 resistance, hygromycin resistance, and the like, for example.

Promoters that can be used for such constructs may be constitutive promoters, or organ-specific or growth stage-specific promoters, and may be selected depending on the host, the necessary amount of expression, the organs in which expression is particularly desired or the growth stage. A preferred embodiment of the present invention uses a powerful promoter that is expressed in a way which is non-specific to organs and growth stages, and one example of such a promoter is the CaMV35S promoter. Organ-specific promoters that can be used include the phaseolin gene promoter and the patatin gene promoter. The most desirable embodiment of the present invention uses a construct that drives the glutamate dehydrogenase gene or aspartate aminotransferase gene by means of a strong structural promoter, such as the CaMV35S promoter.

There are no particular limits on the gene introduction method used in the present invention, and a method known to those skilled in the art for introducing genes into plant cells or plant bodies can be selected according to the host. For example, one embodiment of the present invention employs a gene introduction method using Agrobacterium. A binary vector is preferably used in this transforming system. When using Agrobacterium, the nucleic acid construct used in gene transformation may also include a T-DNA region flanking the DNA sequence which is to be introduced into the plant cells. In a preferred embodiment, the introduced sequence is inserted between left and right T-DNA border sequences. The suitable design and construction of a transformation vector based on such T-DNA is well known to those skilled in the art. The conditions for infecting a plant with Agrobacterium having such a nucleic acid construct are also well known to those skilled in the art. Regarding such techniques and conditions, see Shujunsha Pub., Saibou Kougaku Supplement, “Experimental Protocols in Model Plants: Rice and Arabidopsis thaliana” (1996).

Other gene introduction methods can be used in the present invention. Examples of such gene introduction methods include DNA protoplast introduction using polyethylene glycol or calcium, protoplast transformation using electroporation, and introduction using a particle gun, and the like.

There are no particular limits on the plant that is genetically manipulated in this way, and it is desirable to use a species which is easy to transform and for which a regenerative system has been established, except for when transformation is performed using the whole plant. In addition to the aforementioned properties, a suitable plant which can be used in the present invention is one for which mass cultivation techniques have been established so as to reduce the environmental burden of agriculture. Examples of plants which are suited to the present invention include all plants in the Brassica family, as well as tomatoes, potatoes, corn, wheat, rice, sugar cane, soy beans, sorghum, and the like. Examples of plants that are difficult to supply with large amounts of nitrogen include trees and fruit trees, such as poplar, eucalyptus, and apple trees. The activities and properties of GDHs and ASPCs from these plants are equivalent to those of Aspergillus nidulans-derived GDH (SEQ ID Nos. 1 and 2) and E. coli K-12 strain-derived ASPC (SEQ ID Nos. 19 and 20), which is used in one embodiment of the present invention. Consequently, 2-OG content in these plants can by increased by introducing the Aspergillus nidulans-derived GDH gene (SEQ ID NO: 1) or E. coli-derived ASPC gene (SEQ ID NO: 19). A GDH gene or ASPC gene derived from any of these plants could also be used in the same way in the present invention. There are no particular limits on what organs or cells are subjected to genetic manipulation as described above, and they can be selected depending on the host, the genetic introduction method, and the like. Examples include organ explant, pollen, cultured cells, germ, plant bodies, and the like, but are not limited to these.

Plant cells or the like which have been manipulated as described above are selected for transformation. Selection can be based on expression of a marker gene which is present on the chosen nucleic acid construct. For example, when the marker gene is a drug resistance gene, the manipulated plant cells, or the like, can be cultured or grown on a medium which contains a suitable concentration of an antibiotic, herbicide, or the like. If the marker gene is a beta-glucuronidase gene, luciferase gene, or the like, the transformant can be selected by screening for its activity. When the transformant identified in this way is not a plant body but a protoplast, callus, explant, or the like, it can be regenerated into a whole plant body. A method known to someone with skill in the art can be used for regeneration, depending on the host plant.

The resulting whole plant can be cultivated by ordinary methods or under the same conditions as a non-transformant, and a variety of molecular biology techniques can be used in addition to the aforementioned selection based on marker genes to identify transformed plants comprising the nucleic acid construct of the present invention. For example, Southern hybridization or PCR can be used to detect the presence or absence and the structure of recombinant DNA-inserted fragments. Northern hybridization, RT-PCR, or the like can be used to detect and measure RNA transcription products derived from the introduced nucleic acid construct.

Next, expression of the glutamate dehydrogenase gene or aspartate aminotransferase gene by the transformant is evaluated by measuring the amount of protein, mRNA, or enzyme activity for glutamate dehydrogenase or aspartate aminotransferase. For example, the amount of the glutamate dehydrogenase protein or aspartate aminotransferase protein can be evaluated by Western blotting or the like, and the amount of mRNA by Northern blotting or quantitative RT-PCR. Glutarnate dehydrogenase activity in plant extract can be measured by the methods of Ameziane et al (Ameziane, R., Bernhard, K. and Lightfood, D., Plant and Soil 221: 47-57, 2000). Aspartate aminotransferase activity in plant extract can be measured by the methods of Chao et al (Chao, Y. P., Lai, Z. I., Chen, P. and Chen, J. T., Biotechnology Progress 15:453-458, 1999).

A transformed plant in which expression or increased expression of the glutamate dehydrogenase gene or aspartate aminotransferase gene has been confirmed is further evaluated for 2-OG content. 2-OG content can be assayed, for example, by preparing a plant extract by crushing all or a portion of the transformed plant body. The plant extract can be prepared and the 2-OG assayed by the methods of Usuda (Usuda, H., Plant Physiol. 78:859-864, 1985) for example.

If the 2-OG content of the above-ground parts (for example, the leaves, stems, or both, the floral organs or part of the fruit) or the below-ground parts (for example, the roots or tubers) under normal cultivation conditions and under conditions where nitrogen is limited to a level below normal cultivation conditions is increased 1.2-fold or more as compared with the original line into which the gene has been introduced, it is considered that the 2-OG content is increased.

A transformed plant in which the increased 2-OG content has been confirmed in this way can be cultivated under conditions where nitrogen is limited to below normal cultivation conditions during the cultivation period, and its growth or yield can be evaluated. “Conditions where nitrogen is limited to below normal cultivation conditions” as used herein refers to a condition supplying an amount of nitrogen which is less (for example, such as 70% or less, or preferably 50% or less) than the standard amount of nitrogen, or in other words the standard nitrogen application rate, which is normally used in cultivating each plant. In particular, according to the method of the present invention, a growth and/or yield which is equivalent to or greater than that achieved under cultivation conditions while applying the standard amount of nitrogen is achieved under cultivation conditions in which an amount of nitrogen ranging between below the lower limit of the standard amount and 1/10-fold the lower limit is provided. For example, standard amounts of nitrogen application are established for individual crops. In the case of potatoes, for example, the standard amount of nitrogen is 7 kg per 10 a (JRT Japanese Society of Root and Tuber Crops home page: http://www.jrt.gr.jp/mini/pm_index.html; Minoru Yoshida, Enjoy Learning All About Potatoes, Nobunkyo, 1988), while it is 5 to 15 kg per 10 a for rice (A Manual of Experiments for Plant Biology, Ed. Kokichi Hinata and Teruyoshi Hashiba, Soft Science Publications, 1995; Nara Agricultural Technology Center home page, http://www.naranougi.jp/sehi-kijyun/sehikijyun-index.html). In Arabidopsis thaliana and other experimental plants, a medium comprising 5 mM or more nitrate is considered suitable for growth and development (Martinez-Zapater, J. M. and Salinas, J. Ed., Methods in Molecular Biology, Arabidopsis thaliana Protocols, Humana Press, New Jersey, 1998). Such a standard nitrogen application rate or standard nitrogen amount in a medium is well known to someone with skill in the art.

The transformed plant is cultivated under conditions with nitrogen limited below normal cultivation conditions or in other words, as described above, with an amount of nitrogen in the range between below the lower limit of the standard nitrogen application rate and 1/10 the lower limit, and its growth and/or yield is evaluated. Growth can be evaluated by measuring the above-ground parts or the plant length, number of leaves, or fresh weight or dry weight of the plant as a whole. Improved growth means that at least one of the parameters regarding a whole plant body or a part of a plant body (such as the plant length, number of leaves, fresh weight or dry weight) is 1.2-fold or more as compared with that of a control plant cultivated around the same time under the standard nitrogen application rate. Yield can be evaluated based on the harvested part of the plant, such as the number or individual weight of the tubers for a potato or of the seeds for rice, or the total weight of the harvested parts. Consequently, improved (or increased) yield means that this harvested part has 1.2-fold or more as compared with that of a control plant which is cultivated around the same time under the standard nitrogen application rate.

Once a transformed plant which has better growth and yield than a untransformed control plant under conditions in which nitrogen use is limited to below normal cultivation conditions has been identified, the genetic stability of its characteristics is then investigated. To this end, the plant can be raised, cultivated, and the seeds are collected under normal conditions, and characteristics and their segregation are analyzed in the progeny. The presence or absence, location, expression, and the like of the introduced nucleic acid construct in the progeny can be analyzed in the same way as for the first-generation (T1 generation) transformant.

In a transformed plant the growth and yield of which are increased under conditions with nitrogen limited to below normal cultivation conditions, or in other words a transformed plant the growth of which is no longer inhibited under nitrogen-limited conditions, a sequence derived from a nucleic acid construct incorporated into the introduced genome may be either hemizygous or homozygous, and either a hemizygote or homozygote can be derived in the progeny by cross-breeding, or the like, as necessary. The sequence derived from the nucleic acid construct integrated into the genome is isolated in the progeny by Mendelian genetics. Consequently, a homozygous plant is preferably used for obtaining descendent plants which have stable characteristics.

In most cases, the transformant has an exogenous gene inserted in only one genetic locus, but multi-copy transformants with insertions in multiple gene loci are not unusual. A single-copy transformant is preferred in the present invention for purposes of stability and the like of the introduced gene. For example, a single-copy transformant with the introduced gene in a single gene locus can be selected by investigating the segregation ratio of kanamycin resistance in T2 (the second generation). If T1 is hemizygous and the introduced gene is in a single gene locus, kanamycin resistance and sensitivity will be segregated 3:1 in T2 according to Mendel's law. When there are multiple copies of the introduced gene, the frequency of the resistant transformant increases. Consequently, a single-copy transformant can be obtained by plating the resulting T2 seeds in medium containing kanamycin, selecting a line which exhibits a 3:1 segregation ratio, and selecting those transformants in which the introduced gene is thought to be present in a single gene locus.

The resulting transformed plant can be cultivated under the same conditions as a naturally-occurring plant of the same species, and, when cultivated under conditions where the nitrogen supply is limited to the level below normal cultivation conditions, can provide a harvest equal to or greater than that obtained from a untransformed plant. Moreover, seeds can be obtained from a transformed plant prepared in this way. Seeds can be easily obtained by the same methods used for an untransformed plant of the same species. If necessary, the resulting seeds can be stored, sterilized, and rid of harmful insects by normal methods known to those with skill in the art.

The 2-OG content of a plant can thus be increased by gene introduction, and the 2-OG content of the plant can also be further increased by applying the amino acid proline to the leaves. Specifically, an aqueous solution of 20 mg/l to 500 mg/l of proline can be sprayed or otherwise applied together with a suitable spreader to the leaf surfaces of the plant. The 2-OG content of a plant which has received such foliar application of proline can be evaluated by methods similar to those described above. Moreover, the growth effects under nitrogen-limited conditions can also be evaluated under conditions and by methods similar to those described above.

The present invention is explained more specifically and in more detail below by means of the following non-limiting examples involving the preparation and yield surveys and component analysis of plants manipulated for overexpression of the NADP-GDH gene or aspartate aminotransferase gene, and by the following non-limiting examples involving yield surveys and component analysis of plants which had received foliar application of proline.

### EXAMPLES

### Example 1

### Isolation of Aspergillus nidulans-Derived NADP-Dependent GDH Gene, and Construction of Ti Plasmid Vector

(1) Isolation of Aspergillus nidulans-Derived NADP-Dependent GDH Gene

A. nidulans was seeded on potato dextrose agar medium and cultured overnight at 30° C., and the resulting colony was cultured for 2 days in liquid dextrose medium. Total RNA was prepared from the proliferated cells.

mRNA was purified using a Poly(A) Quick mRNA Isolation Kit (Stratagene), and First-strand cDNA was prepared using a First-strand cDNA Synthesis Kit (Amersham Bioscience). A PCR reaction was performed using the prepared First-strand cDNA as the template. The PCR reaction conditions were 35 cycles of 3 minutes at 94° C., 45 seconds at 94° C., 30 seconds at 59° C. and 90 seconds at 72° C., followed by 10 minutes at 72° C., using a Perkin-Elmer PCR system 2400. The primers used were 5′-TCT AGA ATG TCT AAC CCC CTT GTT GAG-3′ (SEQ ID NO: 5) and 5′-GAG CTC TCA CCA CCA GTC ACC CTG GTC-3′ (SEQ ID NO: 6). A roughly 1.4 kbp band was confirmed, matching the size of the expected gene. The resulting PCR product was cloned using a TA Cloning Kit (Invitrogen). The nucleotide sequence of the resulting clone was determined with a sequencer (ABI 377A). This matched the known A. nidulans-derived NADP-dependent GDH gene, and was given as An-GDH (SEQ ID NO: 17).

(2) Construction of Ti Plasmid Vector

A nucleotide sequence encoding a mitochondrial transit peptide was added to the resulting gene. This was a 5′-end sequence from the tomato-derived NAD-dependent GDH gene which was obtained by PCR using the primers 5′-CTG CAG ATG AAT GCT TTA GCA GCA AC-3′ (SEQ ID NO: 7) and 5′-TCT AGA TAA ACC AAG AAG CCT AGC TG-3′ (SEQ ID NO: 8). Ligation of the resulting An-GDH gene with the nucleotide sequence encoding the mitochondrial transit peptide was accomplished using the four primers 5′-TCT AGA ATG AAT GCT TTA GCA GCA AC-3′ (SEQ ID NO: 9), 5′-GGG AAG GTT TAG ACA TTA AAC CAA GAA GCC T-3′ (SEQ ID NO: 10), 5′-AGG CTT CTT GGT TTA ATG TCT AAC CTT CCC-3′ (SEQ ID NO: 11) and 5′-GAG CTC TTA CGC CTC CCA TCC TCG AA-3′ (SEQ ID NO: 12). The resulting GDH gene with the added mitochondrial transit peptide sequence was designated as Mtd-An-GDH. This Mtd-An-GDH gene was substituted for the GUS region of the Ti plasmid pIG121-Hm, an Agrobacterium transforming vector. The resulting Ti plasmid was introduced into Agrobacterium tumefaciens EHA101, and used to transform Arabidopsis thaliana and potato plants.

### Example 2

### Development of Potato Transformant

Potatoes (May Queen cultivar) were transformed according to the methods of Gordon et al (Ref: Plant Cell Reports, 1993, 12:324-327). Sterilely-induced microtubers were cut, and tuber discs were prepared, placed on MS agar medium supplemented with 2 mg/l zeatin and 0.1 mg/l indole, and cultured for 24 hours at 25° C. with a 16 hour day length. Agrobacterium comprising the constructed gene was inoculated on YEP medium (10 g/l Bacto Tryptone, 10 g/l yeast extract, 1 g/l glucose) containing 50 mg/l kanamycin and 50 mg/l hygromycin, and shaking cultured overnight at 28° C. The Agrobacterium culture was added to infect the tuber discs that had been cultured for 24 hours. After 10 minutes, the excess Agrobacterium was removed using sterilized filter paper, transferred to the aforementioned dish, and cultured for 24 hours under the same conditions. The tuber discs were then transferred on MS agar medium containing 50 mg/l kanamycin, 300 mg/l cefotaxime hydrochloride, 2 mg/l zeatin, and 0.1 mg/l indoleacetic acid, and the transformants were selected. The regenerated shoots were transferred once again to the aforementioned selection medium, and resistance was confirmed. Shoots that clearly exhibited kanamycin resistance were transplanted to MS agar rooting medium containing 50 mg/l kanamycin and 300 mg/l cefotaxime hydrochloride to induce root regeneration. Shoot apices were cut at least three times from the rooted plant and transplanted to selection rooting medium, kanamycin resistance was confirmed, and chimeras were excluded. The resulting 5 individuals were acclimatized to soil to obtain tubers.

### Example 3

### Production of Arabidopsis thaliana Transformants

Gene introduction into Arabidopsis thaliana was accomplished according to the methods of Bechtold et al (Ref: C. R. Acad. Sci. Paris, Life Science 316:1194-1199, 1993). Arabidopsis thaliana seeds were planted on culture soil, and after 10 days of cultivation at 24° C. with a 16 hour day length, the seedlings were transplanted one by one to individual pieces of rock wool, and cultivated for two more weeks under the same conditions. The plants were pinched as soon as they began to bolt, and were then cultivated for another week. Agrobacterium was shaking cultured for 24 hours at 28° C. in YEP culture containing 50 mg/l kanamycin and 50 mg/l hygromycin, and cells were collected by centrifugation (7,000 rpm, 10 minutes). The cells were suspended in suspension medium for infiltration (½ MS salts, ½ B5 vitamin, 5% sucrose, 0.5 g/l MES, 0.044 μM benzylaminopurine, pH 5.7). After removal of the flowering organs which had already flowered or set fruit, the plants were immersed in the Agrobacterium suspension liquid, and treated in a desiccator under reduced pressure (40 mmHg) for 15 minutes. The treated plants were cultivated for 1 month, and seeds were collected. The seeds were planted on MS agar medium containing 50 mg/l kanamycin and 100 mg/l cefotaxime, and selected. Three of the resulting transformants were further cultivated to the 3^{rd} generation (T3), and the lines that no longer exhibited segregation of kanamycin resistance were used in analysis.

### Example 4

### Confirmation of the Introduced Gene

DNA was extracted from selected individual plants which exhibited kanamycin resistance (5 potato and 3 Arabidopsis thaliana plants) and from non-infected plants. DNA was extracted according to the methods of Honda et al (Ref: Honda and Hirai, 1990 Jpn J Breed 40: 339-348). PCR analysis was performed using the extracted DNA, the An-GDH gene-specific primers 5′-TCT AGA ATG TCT AAC CCC CTT GTT GAG-3′ (SEQ ID NO: 13) and 5′-GAG CTC TCA CCA CCA GTC ACC CTG GTC-3′ (SEQ ID NO: 14), and the primers 5′-CCC CTC GGT ATC CAA TTA GAG-3′ (SEQ ID NO: 15) and 5′-CGG GGG GTG GGC GAA GAA CTC CAG-3′ (SEQ ID NO: 16) for amplifying the NPTII gene in the vector. The reaction consisted of 3 minutes at 94° C. followed by 35 cycles of 45 seconds at 94° C., 30 seconds at 55° C. and 90 seconds at 72° C. followed by 10 minutes at 72° C., using a PCR System 2400 (Perkin-Elmer). The PCR product was electrophoresed on 1% agarose gel, and stained with ethidium bromide.

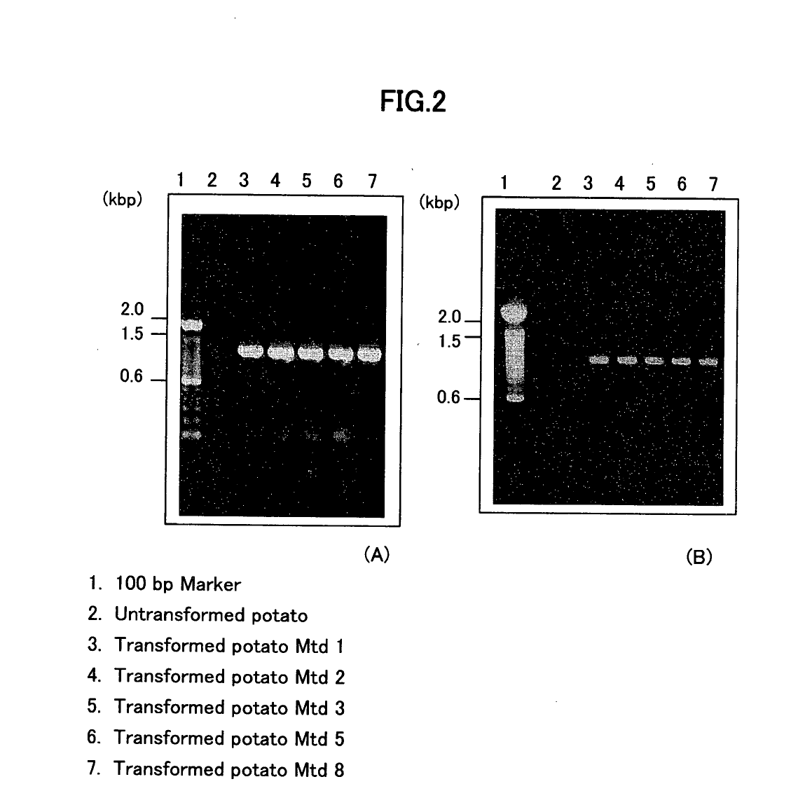

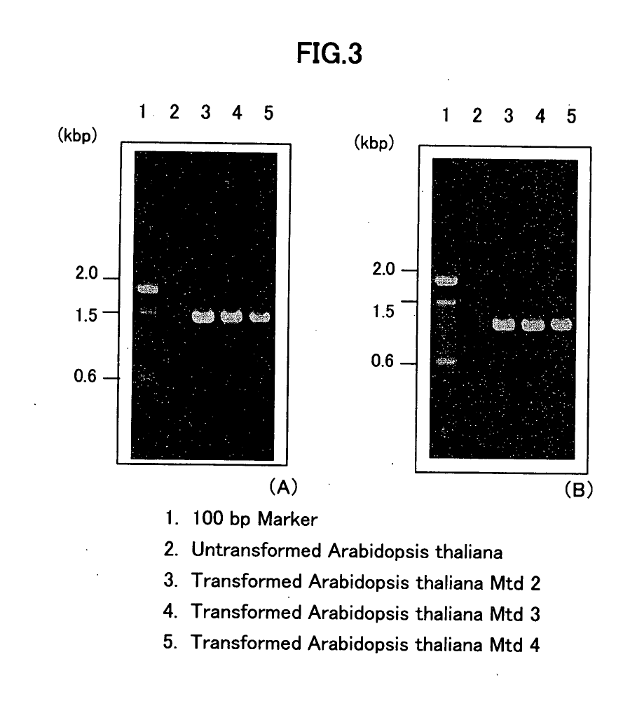

As a result, an An-GDH gene-specific band (about 1.5 kbp) and an NPTII gene-specific band (about 1.1 kbp) were confirmed in the selected transformed potatoes (FIG. 2) and transformed Arabidopsis thaliana (FIG. 3), while no bands were confirmed in the non-infected plants, indicating that a T-DHA region comprising the An-GDH gene had been introduced into the transformed potatoes and the transformed Arabidopsis thaliana.

### Example 5

### Confirmation of Introduced Gene Expression (Northern Analysis)

Expression of the introduced gene was confirmed by Northern analysis using transformed potatoes and transformed Arabidopsis thaliana in which introduction of the An-GDH gene had been confirmed.

RNA was extracted by the SDS-phenol method from transformed potato leaf tissue and tubers and transformed Arabidopsis thaliana leaf tissue. The extracted RNA was electrophoresed on 1.2% agarose gel containing 18% formaldehyde, and stained with ethidium bromide. After being blotted on a nylon membrane (HybondN⁺), it was fixed with UV and hybridized. The full length of the An-GDH gene was used as the probe. The Northern blot and probe were prepared using a DIG-High Prime DNA Labeling and Detection Starter Kit II and PCR DIG Probe Synthesis Kit (Roche Diagnostics).

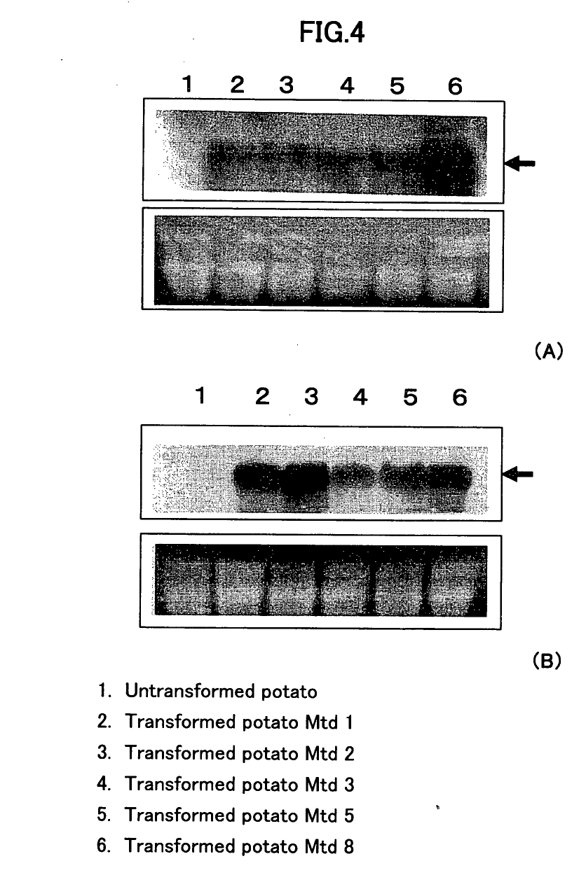

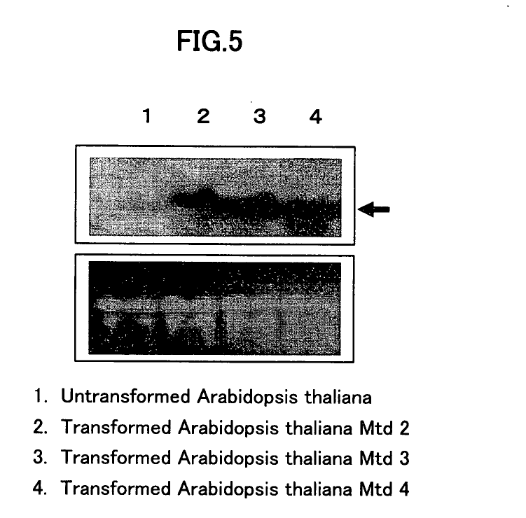

As a result, an An-GDH gene-specific band was confirmed in the leaf tissue and tubers of the transformed potato plants (FIG. 4), showing that the introduced gene was being transcribed and expressed in the leaf tissue and in the tubers of the transformed potato plants. An An-GDH gene-specific band was also confirmed in the leaf tissue of the transformed Arabidopsis thaliana (FIG. 5), indicating that the introduced gene was being transcribed and expressed in the transformed Arabidopsis thaliana leaf tissue. These An-GDH-specific bands were not observed in the untransformed potato or Arabidopsis thaliana plants.

### Example 6

### Expression of Introduced Gene (NADP-GDH Activity)

NADP-GDH activity was measured to confirm expression of the introduced gene using transformed potato and Arabidopsis thaliana plants in which introduction of the An-GDH gene had been confirmed.

Activity was measured according to the methods of Ameziane et al (Ref: Plant and Soil, 2000, 221:47-57). The transformed potato (about 0.2 g) and transformed Arabidopsis thaliana leaf tissue (about 0.2 g) were freeze dried with liquid nitrogen and crushed in a mortar, and 5× by weight of extraction buffer (200 mM Tris (pH 8.0), 14 mM beta-mercaptoethanol, 10 mM L-cysteine-HCl, 0.5 mM PMSF) was added. This was transferred to a centrifugation tube and centrifuged for 30 minutes at 4° C., 12,000 rpm, and the supernatant was ultrafiltered (Millipore, Ultrafree 0.5 filter unit, Biomax-10) and washed 3 times in extraction buffer.

Activity was determined by measuring the reduction in absorbance at a wavelength of 340 nm when a reaction mixture comprising 100 mM Tris (pH 8.5), 20 mM 2-oxoglutaric acid, 10 mM CaCl₂, 0.2 mM NADPH, and 200 mM NH₄Cl was added to the previously extracted raw enzyme solution. The protein concentration in the extracted crude enzyme solution was measured by the Bradford method using bovine serum albumin (BSA) as the standard protein.

As a result, the transformed potato exhibited about 20 to 50 times the activity of the untransformed potato (Table 1). The transformed Arabidopsis thaliana also exhibited about 5 to 12 times the activity of the untransformed Arabidopsis thaliana (Table 2). Since the non-transformants exhibited almost no NADP-GDH activity, the activity was attributed to expression of the introduced An-GDH gene.

TABLE 1
NADP-GDH activity in transformed potatoes and untransformed potato

| | NADP-GDH activity (μmol/min/mg |
|---|---|
| | protein) |
| Non-transformant | 0.11 |
| Transformants: | |
| Mtd 2 | 2.12 |
| Mtd 5 | 4.64 |
| Mtd 8 | 5.85 |

TABLE 2
NADP-GDH activity in transformed Arabidopsis thaliana and
untransformed Arabidopsis thaliana

| | NADP-GDH activity (nmol/min/mg |
|---|---|
| | protein) |
| Non-transformant | 30.0 ± 5.2 |
| Transformants: | |
| Mtd 2 | 393.5 ± 71.3 |
| Mtd 3 | 356.5 ± 70.3 |
| Mtd 4 | 153.9 ± 29.5 |

### Example 7

### 2-oxoglutarate (2-OG), Urea, and Nitrate Content of GDH Gene-Introduced Arabidopsis thaliana and Potato

The 2-OG, urea, and nitrate contents of leaf tissues from transformed potato (Mtd 8) and transformed Arabidopsis thaliana (Mtd) were measured. For the potato, leaf tissue of a vigorously growing plant body that had been cultivated for 1 month in a mixed soil of 500 g Power Soil and 500 g vermiculite was used as the material. The leaf tissue of a plant that had been grown for 3 weeks in PNS medium (containing 5 mM KNO₃) was used for the Arabidopsis thaliana. Extraction was according to the methods of Agarie et al (Ref: Plant Science 2002, 162:257-265). The leaf tissue (about 0.1 g) was first crushed using liquid nitrogen, and then mixed well after addition of 200 μl of 3% HCO₄. An additional 200 μl of 3% HCO₄ was added to the remaining precipitant, and mixed well. After 10 minutes of centrifugation at 12,000 rpm, the supernatant was transferred to the previous tube. 50 to 70 μl of 5M K₂CO₃ was added to the collected supernatant to neutralize the liquid. After the pH of the liquid was confirmed to be neutral using litmus paper, the liquid volume was adjusted to 600 μl with sterile water to obtain the measurement sample. Nitric acid and urea/ammonia F-kits (Roche Diagnostics) were used to measure nitric acid and urea, respectively. The method of Ameziane et al (see previous) was modified for measuring 2-OG content. 20 μl of the extracted sample was added to 475 μl of reaction liquid (0.1 M Tris-HCl (pH 8.5), 1.0 mM CaCl₂, 0.2 mM NADPH, 0.2 M NH₄Cl), absorbance was measured at 340 nm, and 5 μl (10 units) of glutamate dehydrogenase (GDH) was added and reacted for 10 minutes at 37° C. Absorbance was measured again at 340 nm, and 2-OG content was calculated from the difference between absorbance after addition of the enzyme liquid and absorbance before addition of the enzyme.

As a result, the transformed Arabidopsis thaliana had a 2-OG content 2.6 times, urea content 2 times, and a nitrate content 1.2 times that of the untransformed Arabidopsis thaliana.

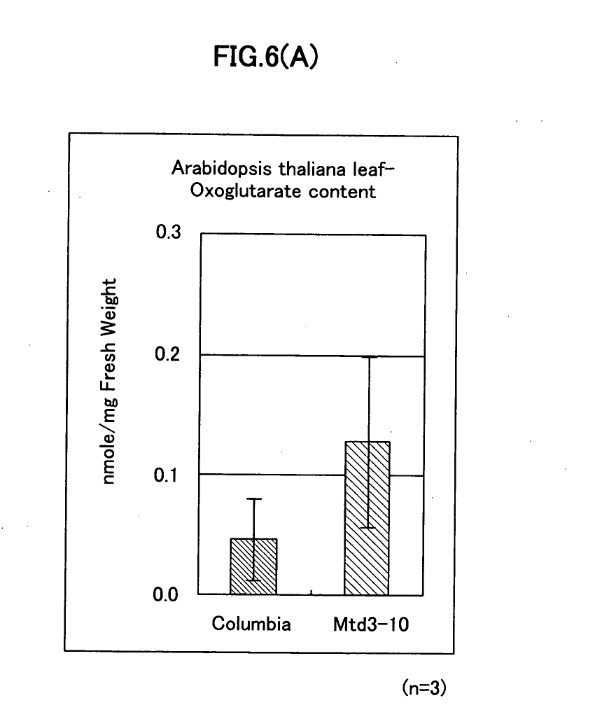

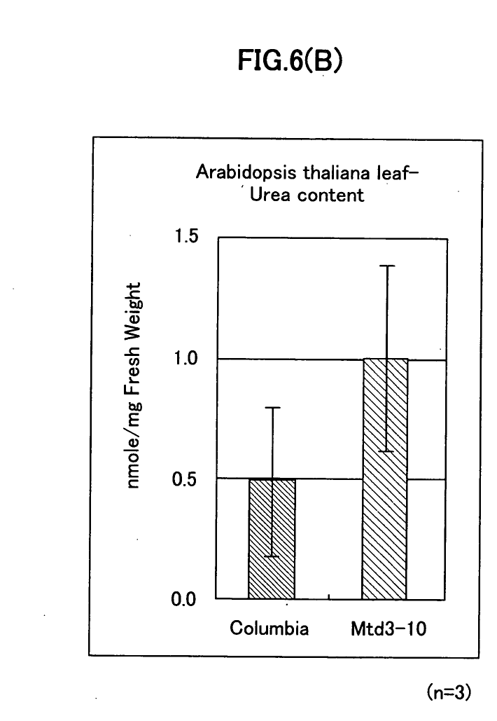

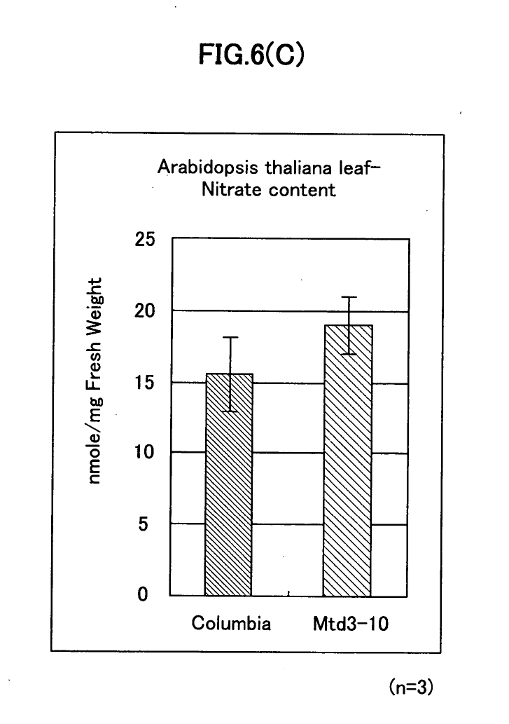

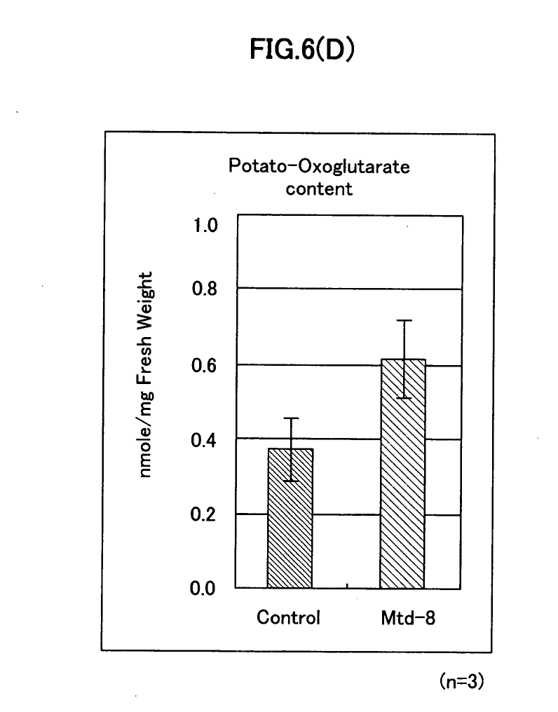

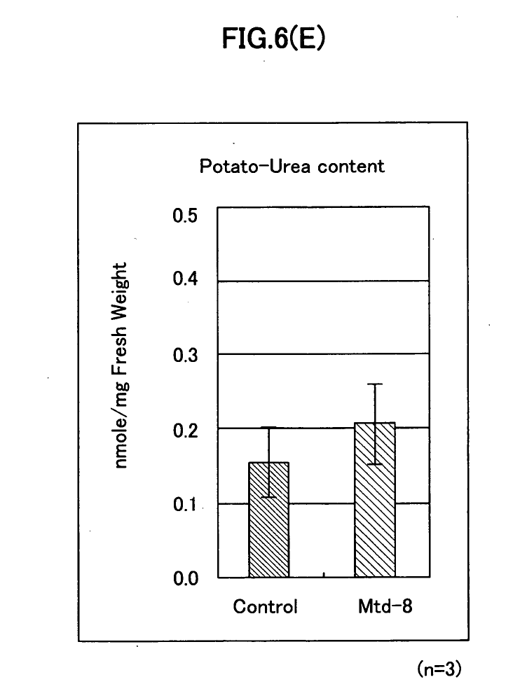

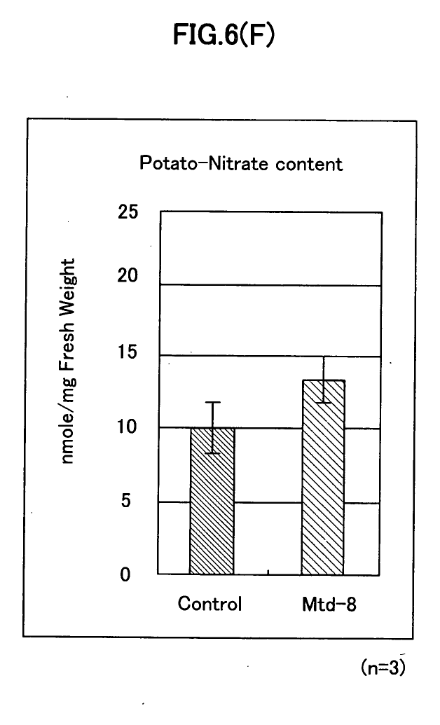

Likewise, the transformed potato had a 2-OG content 1.7 times, urea content 1.5 times, and a nitrate content 1.3 times that of the untransformed potato (FIGS. 6A through 6F).

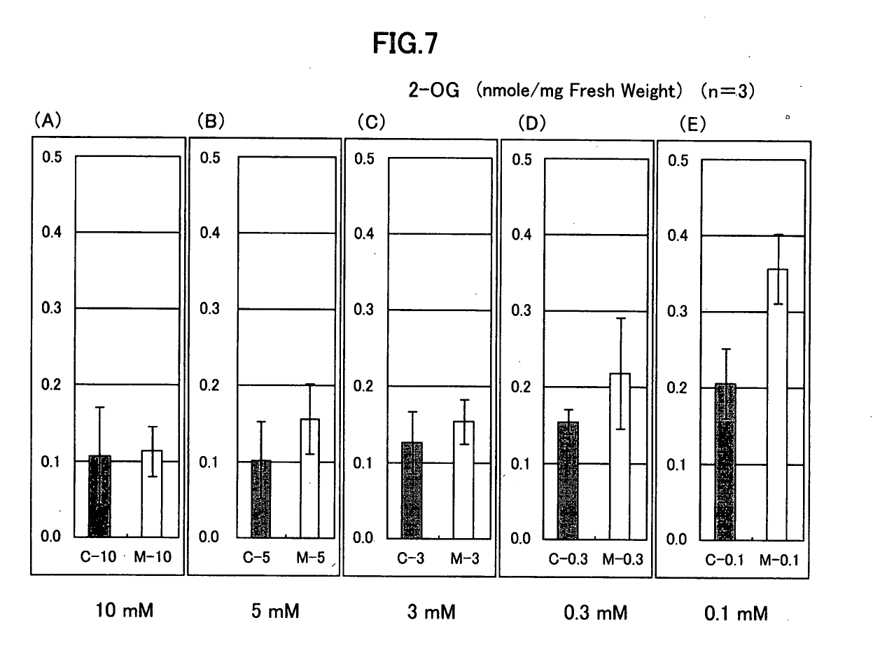

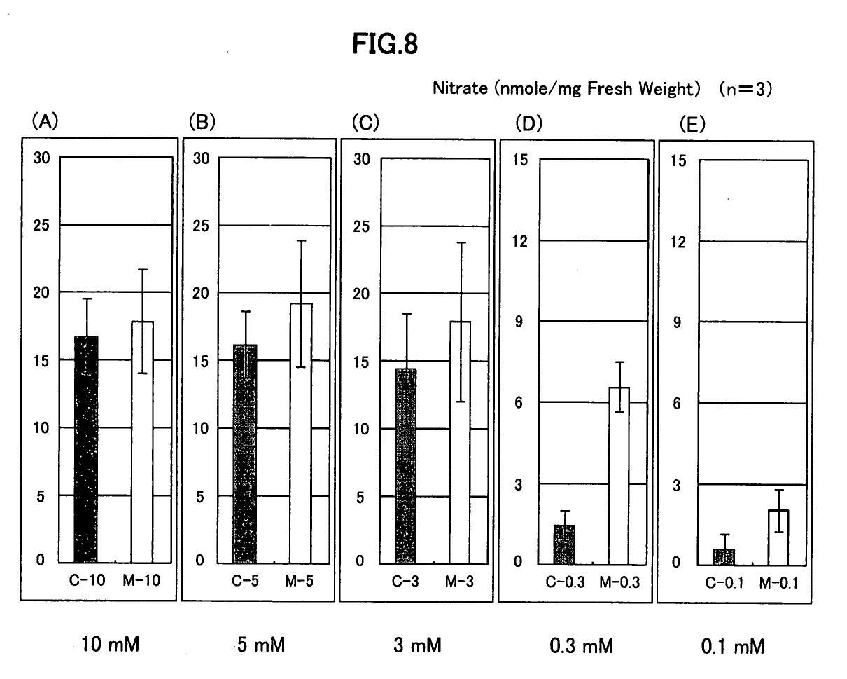

2-OG content and nitrate content were also measured using plant tissue of Arabidopsis thaliana which had been cultured for 3 weeks in medium adjusted to nitrogen concentrations of 10, 5, 3, 0.3 and 0.1 mM. As a result, the difference between the 2-OG content of the transformed Arabidopsis thaliana and the 2-OG content of the untransformed Arabidopsis thaliana was observed to increase as the nitrogen content of the medium decreased (FIG. 7). These results suggest that the introduced GDH functions to break down glutamate and synthesize 2-OG and ammonia. The accumulation of nitrate in the transformed Arabidopsis thaliana and untransformed Arabidopsis thaliana remained roughly steady in a medium containing 3 mM or more of nitrogen, and it was confirmed that under such conditions the accumulation in the transformed Arabidopsis thaliana was at most 1.3 times that in untransformed Arabidopsis thaliana. Under conditions of 0.3 and 0.1 mM nitrogen, however, it was 2.5 to 4.5 times (FIG. 8). These results suggest that a greater amount of nitrate accumulates in transformed Arabidopsis thaliana leaf tissue than in untransformed Arabidopsis thaliana, reflecting an increased efficiency of nitrogen uptake.

### Example 8

### Nitrogen Assimilation-Related Enzyme Activities in the Leaf Tissue of GDH Gene-Introduced Arabidopsis thaliana and Potato

The enzyme activity of NADH-dependent glutamate synthase (NADH-GOGAT), nitrate reductase (NR), and glutamine synthase (GS) was measured in the leaf tissues (about 0.1 g) of transformed potatoes and transformed Arabidopsis thaliana. For the potatoes, the materials were the leaf tissues of vigorously growing plants that had been cultivated for 1 month in a mixed soil of 500 g Power Soil and 500 g vermiculite. The leaf tissues of the plants that had been raised for 3 weeks in PNS medium (containing 5 mM KNO₃) were used for the Arabidopsis thaliana. The crude enzyme solution was extracted by the methods of Groat et al (Ref: Plant Physiol, 1981, 67: 1198-1203). After the leaf tissue had been crushed with liquid nitrogen, 500 μl of ice-cooled extraction buffer (100 mM Mes-NaOH (pH 7.5), 100 mM sucrose, 2% (v/v) mercaptoethanol, 15% ethylene glycol, 0.1% PMSF) was added and mixed gently. Following centrifugation for 20 minutes at 0° C., 12,000 rpm, the supernatant was purified with Ultrafree (Biomax-10K, Millipore Co.). This was washed three times with the same buffer and dissolved in 100 μl of buffer to obtain the enzyme solution.

NADH-GOGAT activity was also measured by the methods of Groat et al (see previous). 500 μl of reaction solution (100 mM K-phosphate (pH 8.2), 100 μM NADH, 2.5 mM 2-oxoglutarate, 10 mM L-glutamine, 1 mM aminooxyacetate) was prepared, and 10 μl of the enzyme liquid was added thereto and mixed. Absorbance was measured for 3 minutes at 340 nm, and the decrease accompanying consumption of NADH was measured.

NR activity was measured by the methods of Ferrario-Mery et al (Ref: Planta, 197, 202:510-521). 490 μl reaction solution (50 mM Mops-KOH (pH 7.8), 1 mM NaF, 10 mM KNO₃, 0.17 mM NADH, and 10 mM MgCl₂ or 5 mM EDTA) was prepared, and 10 μl of enzyme solution was added to each and mixed. 16 minutes later 500 μl of 1% sulfonyl amide (in 3N HCl) was added, and then 10 μl of 2% N-naphthyl-1-ethylene diamine dichlorohydrate was added. Following centrifugation at 12,000 rpm for 5 minutes, absorbance was measured at 540 nm. The value from the reaction with added EDTA was subtracted from the value from the reaction with added MgCl₂ to obtain NR activity.

GS activity was also measured by the methods of Ferrario-Mery et al (see previous). 20 μl of enzyme solution was added to 480 μl of reaction liquid (80 mM glutamate, 20 mM MgSO₄, 1 mM EDTA, 100 mM tricine, 6 mM hydroxylamine, 8 mM ATP), and reacted for 15 minutes at 30° C. 500 μl of 0.37 M FeCl₃, 0.2 M of trichloroacetic acid and a 0.67 N HCl solution were added to stop the reaction, and absorbance was measured at 540 nm.

The protein concentration of the extracted enzyme solution was measured by the Bradford method using bovine serum albumin (BSA) as a standard protein. As a result, the transformed Arabidopsis thaliana had 1.38 times the NADH-GOGAT activity, 1.40 times the NR activity, and 1.25 times the GS activity of the untransformed Arabidopsis thaliana (Table 3). The transformed potato also had 1.33 times the NADH-GOGAT activity, 1.36 times the NR activity, and 1.01 times the GS activity of the untransformed potato (Table 4). These results show that not only do transformed Arabidopsis thaliana and the transformed potatoes have greater nitrogen uptake efficiency than non-transformants, but enzymes involved in nitrogen metabolism are also activated, and the nitrogen metabolism efficiency was also increased in the transformed Arabidopsis thaliana and the transformed potatoes.

TABLE 3
Enzyme activities in the transformed Arabidopsis thaliana (Mtd 3)

| | NADH-GOGAT | NR | GS |
|---|---|---|---|
| Non-transformant (A) | 482.4 ± 86.8 | 1.88 ± 0.22 | 188.3 ± 24.9 |
| Transformant (B) | 667.5 ± 107.5 | 2.63 ± 0.37 | 235.7 ± 28.4 |
| Ratio (B/A) | 1.38 | 1.40 | 1.25 |

(units/mg protein)

TABLE 4
Enzyme activities in the transformed potato (Mtd 8)

| | NADH- | | |
|---|---|---|---|
| | GOGAT | NR | GS |
| Non-transformant (A) | 38.6 ± 4.1 | 0.21 ± 0.04 | 99.0 ± 20.7 |
| Transformant (B) | 51.5 ± 2.4 | 0.29 ± 0.03 | 99.6 ± 8.5 |
| Ratio (B/A) | 1.33 | 1.36 | 1.01 |

(units/mg protein)

### Example 9

### Yield Survey of Transformed Potato

The yields of the transformed potatoes were studied. Seed potatoes from the transformed potatoes and the untransformed potatoes were used in the yield survey. Growth, tuber weight, number of tubers, and the like were determined under cultivation conditions with differing nitrogen concentrations. Two groups were set up—one is for standard conditions of 3 kg of Power Soil (total nitrogen 1.2 g) in an 8.5-liter pot and the other is for low-nitrogen conditions of 300 g of Power Soil (total nitrogen 0.12 g) in a No. 7 pot, with the remainder of the soil being made up of vermiculate (without nutrients). The potatoes were harvested 3 months after being seeded, and above-ground weights, tuber weights, and tuber numbers were measured.

In the 1.2 g nitrogen group, the transformed potatoes had 1.12 to 1.13 times the tuber weight and 1.09 to 1.23 times the number of tubers of the untransformed potatoes (Table 5). In the 0.12 g nitrogen group (nitrogen-limited conditions), the transformed potatoes had 1.29 to 1.35 times the tuber weight and 1.24 to 1.56 the number of tubers of the untransformed potatoes (Table 6).

TABLE 5
Yield survey of transformed potatoes in 1.2 g nitrogen group (n ≧ 8)

| | | Transformant | |
|---|---|---|---|
| | Non-transformant | Mtd 3 | Mtd 8 |
| Above-ground weight | 8.2 ± 1.9 (1.00) | 7.8 ± 2.0 (0.95) | 8.6 ± 0.8 (1.05) |
| (g) | | | |
| Tuber weight (g) | 217.6 ± 19.3 (1.00) | 244.1 ± 20.4 (1.12) | 246.1 ± 21.0 (1.13) |
| Number of tubers | 6.5 ± 1.7 (1.00) | 7.1 ± 2.8 (1.09) | 8.0 ± 2.1 (1.23) |

TABLE 6
Yield survey of transformed potatoes in 0.12 g nitrogen group (n ≧ 8)

| | | Transformant | |
|---|---|---|---|
| | Non-transformant | Mtd 1 | Mtd 8 |
| Above-ground | 11.9 ± 2.1 (1.00) | 14.0 ± 4.0 (1.18) | 13.5 ± 2.4 (1.13) |
| weight (g) | | | |
| Tuber | 41.0 ± 7.0 (1.00) | 52.7 ± 7.9 (1.29) | 55.4 ± 6.5 (1.35) |
| weight (g) | | | |
| Number of | 2.5 ± 0.5 (1.00) | 3.9 ± 1.0 (1.56) | 3.1 ± 1.1 (1.24) |
| tubers | | | |

### Example 10

### Growth Survey of Transformed Arabidopsis thaliana

Growth was studied using transformed Arabidopsis thaliana (Mtd 3). The Arabidopsis thaliana were sown on PNS medium (nitrogen source: nitrate nitrogen only) supplemented with varying KNO₃ concentrations of 0.1, 0.3, 3, and 5 mM and 1% sucrose using 8 cm Thermo Petri dishes, 8 plants per dish. 3 weeks after sowing, the above-ground weights were measured. The data represent the total above-ground weights of all 8 plants, presented as the mean and deviation for 3 Petri dishes in each group. As a result, the transformed Arabidopsis thaliana grew better under low-nitrogen conditions than did the untransformed Arabidopsis thaliana, exhibited a growth 1.1 to 1.14 times that of the untransformed Arabidopsis thaliana as fresh weight (Table 7).

TABLE 7
Fresh weights (g) of transformed Arabidopsis thaliana and untransformed Arabidopsis thaliana
grown in PNS medium (with 1% sucrose) comprising various KNO₃ concentrations

| KNO₃ concentration | 0.1 mM | 0.5 mM | 1.0 mM | 5.0 mM |
|---|---|---|---|---|
| Non-transformant (A) | 0.0704 ± 0.0073 | 0.1265 ± 0.0054 | 0.2026 ± 0.0037 | 0.5895 ± 0.0032 |
| Transformant (B) | 0.0800 ± 0.0073 | 0.1441 ± 0.0054 | 0.2232 ± 0.0037 | 0.6101 ± 0.0032 |
| Ratio (B/A) | 1.14 | 1.14 | 1.10 | 1.03 |

### Example 11

### Foliar Application of an Aqueous Proline Solution to Potato Plants

The effects of foliar application of a proline solution on growth and yield were investigating using potatoes (May Queen cultivar). Potato tubers were placed in No. 7 pots containing 1.5 kg of Power Soil (Kureha Chemical). The nitrogen fertilizer content of the Power Soil was shown in Table 8. Under normal cultivation conditions, about 5 g of nitrogen fertilizer is used as base fertilizer; while in this cultivation test, cultivation was under low-nitrogen conditions.

Three foliar application groups were prepared—a spreader group (Approach B1, Kao), a urea group (spreader+0.87 mM urea), and a proline group (spreader+1.75 mM proline), and about 20 ml per plant was sprayed once a week beginning about a month after planting (immediately before flowering). The leaves were sprayed 6 times (6 weeks), and harvested 3 months after planting.

TABLE 8
Nitrogen fertilization conditions for the potato cultivation test

| | |
|---|---|
| Total nitrogen | 0.6 g |
| Nitrate-nitrogen | 0.075 g |
| Ammonia-nitrogen | 0.525 g |

### Example 12

### Measurement of Free Proline Content and 2-oxoglutarate (2-OG) Content of Potato Leaf Tissues After Foliar Application of Proline

After potato leaf surfaces had been sprayed with aqueous solutions of a spreader, urea and proline, the free proline content and 2-OG content were measured over time. The 3^{rd} leaf from the apices was sampled 1, 3, 5, and 8 hours after foliar application, and washed thoroughly with water as the samples. After crushing with liquid nitrogen, 80% ethanol, which had been heated to 80° C. and was equal to 5 times the fresh weight, was added, and incubated for 20 minutes at 80° C. Following centrifugation for 10 minutes at 12,000 rpm, the supernatant was transferred to a separate tube. 80% ethanol was added again, followed by incubation for 20 minutes at 80° C. and centrifugation, and the supernatant was collected and extracted in the same way a total of 3 times. The ethanol extract was freeze dried, and 300 μl sterile water and 200 μl ethyl ether were added and mixed well and centrifuged for 10 minutes at 12,000 rpm. After removal of the upper, ethyl ether layer, 200 μl of the water layer was transferred to a separate tube and once more freeze dried. After being dissolved in 200 μl of 0.02 N hydrochloric acid, this was filtered with a 0.22 μm filter. The resulting extract was analyzed with a high-speed amino acid analyzer (L8800 Hitachi). The 2-OG content was also measured using similar plant samples. The extraction and the determination of the content of 2-OG was conducted according to methods similar to those described in Example 7.

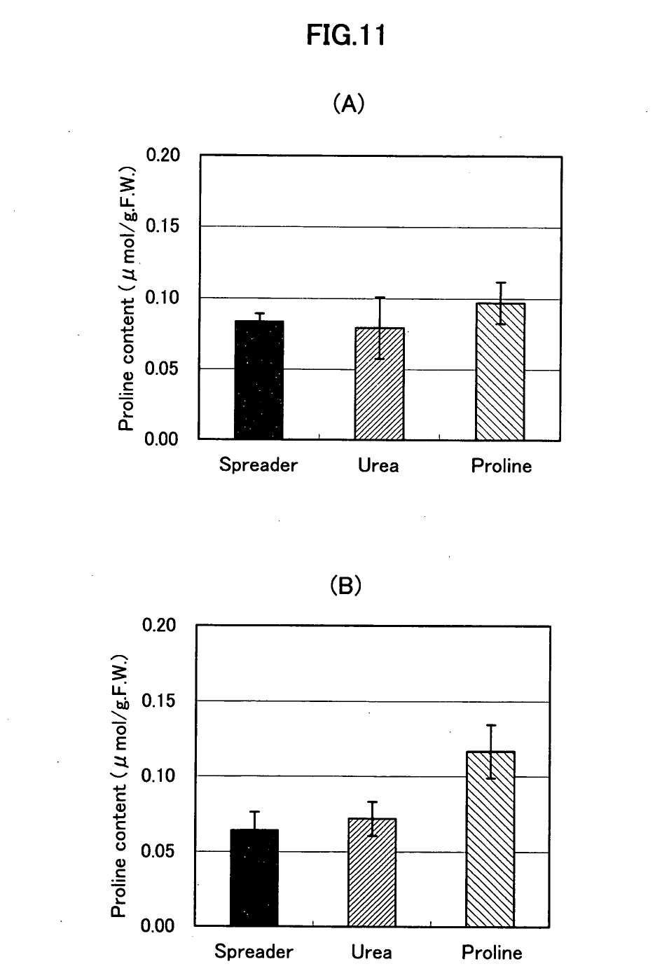

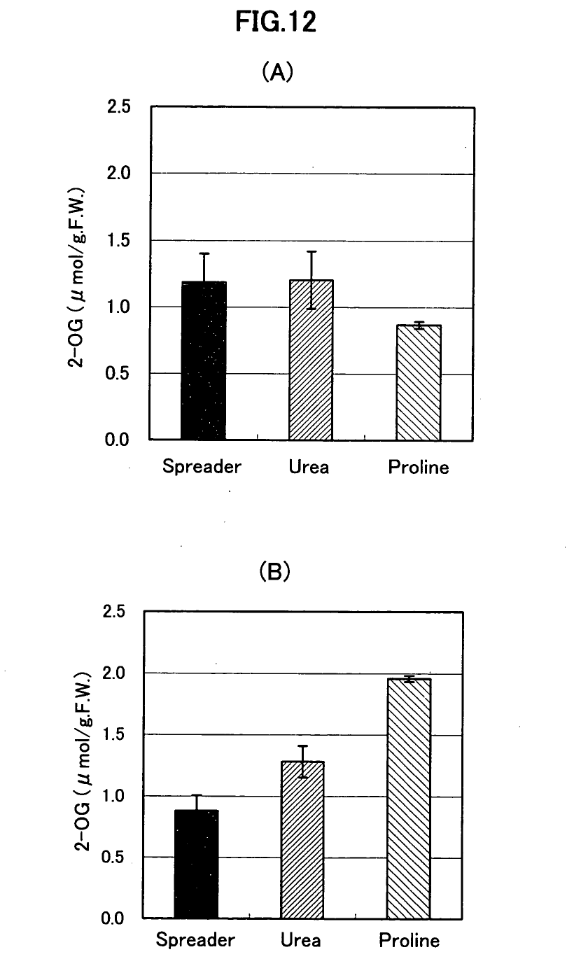

As a result, no significant difference was observed among the three groups in the proline content of the leaf tissue one hour after foliar application, but 5 hours after application the free proline content of the leaf tissue was significantly greater in the proline application group (FIG. 11). It was thought that the proline sprayed on the leaf surfaces had been incorporated into the leaf tissue. Similarly, when the 2-OG content in leaf tissue was measured no significant difference was observed among the three groups in 2-OG content in leaf tissue 1 hour after foliar application, but 24 hours after application the 2-OG content of the leaf tissue was significantly greater in the proline application group (FIG. 12). It was thought that the proline which had been sprayed on the leaf surfaces had been incorporated into the leaf tissue, and then metabolized into 2-OG and entered into the TCA cycle.

### Example 13

### Yield Survey of Potatoes after Foliar Application of Proline

Aqueous solutions of a spreader, urea and proline were applied 6 times at one-week intervals to the leaf surfaces of potato plants 1 month after planting. These were cultivated for 3 months after planting, with 6 pots in each group and one stalk per plant. The tubers were harvested from each pot, and the tuber weight and number of tubers per pot was measured.

As a result, the tuber weight per pot was 145.8 g per pot in the spreader group, 140.1 g in the urea group and 158.4 g in the proline group, so the proline group had 1.1 times the tuber weight of the spreader group. The number of tubers was 8.2 in the spreader group, 6.8 in the urea group, and 8.2 in the proline group, so no difference was seen in tuber numbers (Table 9). These results indicate that tuber weight is increased by foliar application of proline.

TABLE 9
Yield of proline-sprayed potatoes (n = 6)

| | Spreader | Urea | Proline |
|---|---|---|---|
| Total tuber weight | 145.8 ± 16.3 | 140.1 ± 10.6 | 158.4 ± 18.0 |
| (per plant) | | | |
| Ratio | 1.00 | 0.96 | 1.10 |
| Total number of tubers | 8.2 ± 1.9 | 6.8 ± 1.6 | 8.2 ± 1.0 |
| (per plant) | | | |
| Ratio | 1.00 | 0.83 | 1.00 |

### Example 14

### Production of Transformed Arabidopsis thaliana into which an E. coli-Derived Aspartate Aminotransferase (ECASPC) Gene is Introduced

An ECASPC gene was isolated based on the sequence data (GenBank accession NO: X03629) for an aspartate aminotransferase gene (ECASPC) already reported in E. coli (nucleotide sequence: SEQ ID NO: 19; amino acid sequence: SEQ ID NO: 20). A mitochondrial transit peptide sequence was added to this gene as described in Example 1 (mtdECASPC). This gene was substituted for the GUS region of the plant-transforming Ti-plasmid vector pBI121, and transformed into Agrobacterium tumefaciens EHA105. The mtdECASPC gene was introduced into Arabidopsis thaliana by the methods described in Example 3 using this Agrobacterium. This was then sowed in medium containing kanamycin, resistant individuals were selected (T1 generation), and a line in which kanamycin resistance and sensitivity were segregated 3:1 was obtained in the next generation (T2 generation). In the following generation, four lines with all individuals exhibiting kanamycin resistance (T3 generation) were selected (mtdECASPC2-2, mtdECASPC6-2, mtdECASPC8-1 and mtdECASPC9-1). The introduced gene was confirmed in the resulting transformants by genome PCR as in Example 4. Transcription and expression of the introduced gene were also analyzed by RT-PCR. RNA was extracted from the Arabidopsis thaliana leaf tissue using a Plant RNeasy Mini-Kit (Qiagen). RT-PCR was performed using a RNA-PCR Kit (TaKaRa) and ECASPC-specific primers. The reaction consisted of 30 cycles of 1 minute at 94° C., 1 minute at 55° C. and 2 minutes at 72° C., followed by 1% agarose gel electrophoresis and ethidium bromide staining.

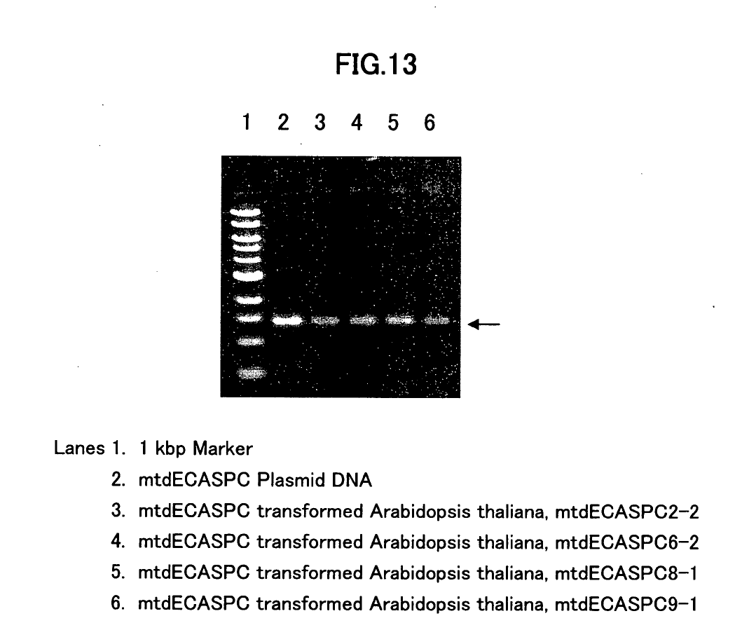

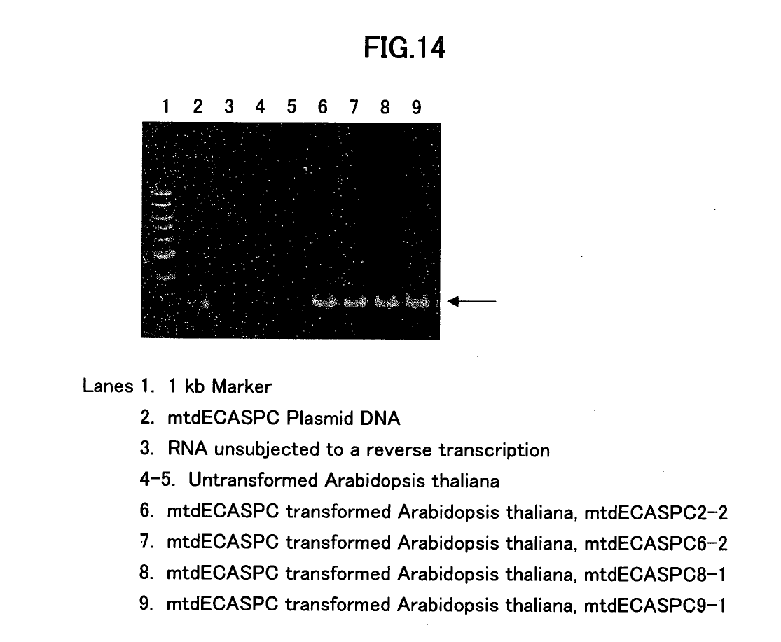

As a result, the transformed Arabidopsis thaliana that exhibited kanamycin resistance produced a band of the same size as that obtained using the template mtdECASPC plasmid DNA as a positive control (FIG. 13). These results suggest that the mtdECASPC gene had been introduced into the genome of the transformed Arabidopsis thaliana which exhibited kanamycin resistance. An mtdECASPC-specific band was also confirmed by RT-PCR in the transformed Arabidopsis thaliana (FIG. 14). Since this band was not seen with untransformed Arabidopsis thaliana or when RNA without the reverse transcription was used as the template, it appears that the genome was not contaminated. That is, these results suggest that the introduced mtdECASPC gene was being transcribed within the transformed Arabidopsis thaliana.

### Example 15

### Growth Survey of ECASPC Gene-Transformed Arabidopsis thaliana Under Nitrogen-Limited Conditions

Growth was surveyed as in Example 10 using the resulting transformants. PNS having nitrate nitrogen alone as the nitrogen source was used. The KNO₃ concentrations of the media were 5 mM and 10 mM, each medium was supplemented with 1% sucrose. The seeds were sterilely sown, and 10 plants with uniform growth were allowed to remain after 1 week. After 2 more weeks (3 weeks after sowing), the above-ground weights of the 10 plants were measured. The means and deviations for 2 plates are shown in Table 10. As a result, it was shown that Arabidopsis thaliana containing the introduced mtdECASPC gene had much greater above-ground weights than the untransformed Arabidopsis thaliana into which the gene had not been introduced, by a factor of 1.18 to 1.28 in the 5 mM nitrogen group and 1.12 to 1.38 in the 10 mM nitrogen group.

TABLE 10
Growth survey of Arabidopsis thaliana with introduced
mtdECASPC gene

| | 5 mM | 10 mM |
|---|---|---|
| Non-transformant | 335.6 ± 10.5 | 352.2 ± 25.5 |
| Transformants: | | |
| mtdECASPC2-2 | 405.4 ± 0.8 | 394.7 ± 10.3 |
| mtdECASPC6-2 | 394.7 ± 7.7 | 436.4 ± 3.4 |
| mtdECASPC8-1 | 430.3 ± 5.8 | 484.6 ± 7.0 |
| mtdECASPC9-1 | 420.6 ± 13.2 | 467.5 ± 2.2 |

(mg, n = 2)

### Example 16

### Measurement of 2-OG Content of mtdECASPC Gene-Transfected Arabidopsis thaliana

2-OG content was measured using the four lines of mtdECASPC gene-transfected Arabidopsis thaliana selected in Example 14. The above-ground parts of plants grown for 2 weeks at 24° C. with a 16-hour day length from seeds which were sown sterilely in PNS medium containing 5 mM KNO₃ and 1% sucrose were used. 2-OG was extracted and its content measured by methods similar to those described in Example 7.

As a result, the 2-OG content of the above-ground tissue of the transformed Arabidopsis thaliana with the introduced mtdECASPC gene was greater by a factor of 1.27 to 1.93 than that of the untransformed Arabidopsis thaliana into which the gene had not been introduced. The results are extremely similar to the results obtained for transformed plants with the introduced GDH gene.

TABLE 11
2-OG content of mtdECASPC gene-transfected Arabidopsis thaliana

| | 2-OG content | |
|---|---|---|
| | (μmol/g.F.W.) | Ratio |
| Non-transformant | | |
| Transformant | | |
| mtdECASPC2-2 | 0.254 ± 0.029 | 1.35 |
| mtdECASPC6-2 | 0.363 ± 0.085 | 1.93 |
| mtdECASPC8-1 | 0.239 ± 0.066 | 1.27 |
| mtdECASPC9-1 | 0.268 ± 0.080 | 1.42 |

(n > 3)

While the invention has been described in detail with reference to preferred embodiments thereof, it will be apparent to one skilled in the art that various changes can be made, and equivalents employed, without departing from the scope of the invention. Each of the aforementioned documents is incorporated by reference herein in its entirety.

1. A method for producing a plant which exhibits improved growth and/or improved yield under nitrogen-limited cultivation conditions, comprising increasing the 2-oxoglutarate (2-OG) content of the plant.

2. The method of claim 1, wherein said 2-oxoglutarate content is increased by introducing and expressing a glutamate dehydrogenase (GDH) gene into said plant.

3. The method of claim 1, wherein said 2-oxoglutarate content is increased by spraying proline on a leaf of said plant.

4. The method of claim 1, wherein said 2-oxoglutarate content is increased by introducing and expressing an ECASPC gene into said plant.

5. A method for cultivating a plant which has increased 2-OG content, comprising cultivating said plant under nitrogen-limited conditions.

6. The method of claim 5, wherein a glutamate dehydrogenase (GDH) gene has been introduced into said plant.

7. The method of claim 5, wherein said plant has received foliar application of proline.

8. The method of claim 5, wherein an ECASPC gene has been introduced into said plant.

9. The method according to claim 1, wherein said nitrogen-limited cultivation conditions comprise an amount of nitrogen which is between less than the lower limit and 1/10-fold the lower limit of the standard application amount of nitrogen fertilizer.

10. The method according to claim 5, wherein said nitrogen-limited conditions comprise an amount of nitrogen which is between less than the lower limit and 1/10 the lower limit of the standard application amount of nitrogen fertilizer.
